SEQUENCE LISTING (3 sequences)

SEQ ID NO: 1 - protein, 808 aa, Arabidopsis thaliana
MANAERMITRVHSQRERLNETLVSERNEVLALLSRVEAKGKGILQQNQIIAEFEALPEQTRKKLEGGPFFDLLKSTQEAIVLPPWVALAVRPRPGVWEYLRVNLHALVVEELQPAEFLHFKEELVDGVKNGNFTLELDFEPFNASIPRPTLHKYIGNGVDFLNRHLSAKLFHDKESLLPLLDFLRLHSHQGKNLMLSEKIQNLNTLQHTLRKAEEYLAELKSETLYEEFEAKFEEIGLERGWGDNAERVLDMIRLLLDLLEAPDPSTLETFLGRVPMVFNVVILSPHGYFAQDNVLGYPDTGGQVVYILDQVRALEIEMLQRIKQQGLNIKPRILILTRLLPDAVGTTCGERLERVYDSEYCDILRVPFRTEKGIVRKWISRFEVWPYLETYTEDAAVELSKELNGKPDLIIGNYSDGNLVASLLAHKLGVTQCTIAHALEKTKYPDSDIYWKKLDDKYHFSCQFTADIFAMNHTDFIITSTFQEIAGSKETVGQYESHTAFTLPGLYRVVHGIDVFDPKFNIVSPGADMSIYFPYTEEKRRLTKFHSEIEELLYSDVENDEHLCVLKDKKKPILFTMARLDRVKNLSGLVEWYGKNTRLRELVNLVVVGGDRRKESKDNEEKAEMKKMYDLIEEYKLNGQFRWISSQMDRVRNGELYRYICDTKGAFVQPALYEAFGLTVVEAMTCGLPTFATCKGGPAEIIVHGKSGFHIDPYHGDQAADLLADFFTKCKEDPSHWDEISKGGLQRIEEKYTWQIYSQRLLTLTGVYGFWKHVSNLDRLEHRRYLEMFYALKYRPLAQAVPLAQDD

SEQ ID NO: 2 - protein, 442 aa, Solanum lycopersicum
MATNLRVLMFPWLAYGHISPFLNIAKQLADRGFLIYLCSTRINLESIIKKIPEKYADSIHLIELQLPELPELPPHYHTTNGLPPHLNPTLHKALKMSKPNFSRILQNLKPDLLIYDVLQPWAEHVANEQGIPAGKLLVSCAAVFSYFFSFRKNPGVEFPFPAIHLPEVEKVKIREILAKEPEEGGRLDEGNKQMMLMCTSRTIEAKYIDYCTELCNWKVVPVGPPFQDLITNDADNKELIDWLGTKPENSTVFVSFGSEYFLSKEDMEEIAFALEASNVNFIWVVRFPKGEERNLEDALPEGFLERIGERGRVLDKFAPQPRILNHPSTGGFISHCGWNSVMESIDFGVPIIAMPIHNDQPINAKLMVELGVAVEIVRDDDGKIHRGEIAEALKSVVTGETGEILRAKVREISKNLKSIRDEEMDAVAEELIQLCRNSNKSK

SEQ ID NO: 3 - protein, 458 aa, Stevia rebaudiana
MENKTETTVRRRRRIILFPVPFQGHINPILQLANVLYSKGFAITILHTNFNKPKTSNYPHFTFRFILDNDPQDERISNLPTHGPLAGMRIPIINEHGADELRRELELLMLASEEDEEVSCLITDALWYFAQDVADSLNLRRLVLMTSSLFNFHAHVSLPQFDELGYLDPDDKTRLEEQASGFPMLKVKDIKSAYSNWQIGKEILGKMIKQTKASSGVIWNSFKELEESELETVIREIPAPSFLIPLPKHLTASSSSLLDHDRTVFEWLDQQAPSSVLYVSFGSTSEVDEKDFLEIARGLVDSGQSFLWVVRPGFVKGSTWVEPLPDGFLGERGKIVKWVPQQEVLAHPAIGAFWTHSGWNSTLESVCEGVPMIFSSFGGDQPLNARYMSDVLRVGVYLENGWERGEVVNAIRRVMVDEEGEYIRQNARVLKQKADVSLMKGGSSYESLESLVSYISSL

(19) United States
(12) Patent Application Publication
(10) Pub. No.: US 2021/0095322 A1
(43) Pub. Date: Apr. 1, 2021

(54) HIGH-PURITY STEVIOL GLYCOSIDES

(71) Applicant: PureCircle USA Inc., Chicago, IL (US)

(72) Inventors: Avetik Markosyan, Yerevan (AM); Saravanan A/L Ramandach, Seremban (MY); Mohamad Afzaal Bin Hasim, Kuala Lumpur (MY); Khairul Nizam Bin Nawi, Nilai (MY); Siew Yin Chow, Klang (MY); Siddhartha Purkayastha, Chicago, IL (US); Marcia Petit, Chicago, IL (US)

(73) Assignee: PureCircle USA Inc., Chicago, IL (US)

(21) Appl. No.: 16/981,687

(22) PCT Filed: Mar. 15, 2019
(86) PCT No.: PCT/US19/22581

§ 371 Date: Sep. 16, 2020

Related U.S. Application Data
(63) Continuation-in-part of application No. PCT/US2018/026920, filed on Apr. 10, 2018.
(60) Provisional application No. 62/644,065, filed on Mar. 16, 2018.
(60) Provisional application No. 62/644,407, filed on Mar. 17, 2018.

(51) Int. Cl. C12P 19/56; C12N 9/10; C07H 15/256; A23L 27/30; A23L 27/00; A23L 2/60; A23C 9/156

(52) U.S. Cl. CPC C12P 19/56; C12N 9/1062; C12N 9/1051; C07H 15/256; A23V 2002/00; A23L 27/88; A23L 2/60; A23C 9/156; A23L 27/36

(57) ABSTRACT

Methods of using highly purified rebaudioside AM are described. The methods include utilizing enzyme preparations and recombinant microorganisms for converting various staring compositions to target steviol glycosides. The highly purified rebaudioside AM is useful as flavor enhancer, sweetness enhancer, and foaming suppressor in edible and chewable compositions such as any beverages, confectioneries, bakery products, cookies, and chewing gums.

### TECHNICAL FIELD

The present invention relates to compositions comprising steviol glycosides, including highly purified steviol glycoside compositions, and processes for making the same.

### BACKGROUND OF THE INVENTION

High intensity sweeteners possess a sweetness level that is many times greater than the sweetness level of sucrose. They are essentially non-caloric and are commonly used in diet and reduced-calorie products, including foods and beverages. High intensity sweeteners do not elicit a glycemic response, making them suitable for use in products targeted to diabetics and others interested in controlling for their intake of carbohydrates.

Steviol glycosides are a class of compounds found in the leaves of Stevia rebaudiana Bertoni, a perennial shrub of the Asteraceae (Compositae) family native to certain regions of South America. They are characterized structurally by a single base, steviol, differing by the presence of carbohydrate residues at positions C13 and C19. They accumulate in Stevia leaves, composing approximately 10%-20% of the total dry weight. On a dry weight basis, the four major glycosides found in the leaves of Stevia typically include stevioside (9.1%), rebaudioside A (3.8%), rebaudioside C (0.6-1.0%) and dulcoside A (0.3%). Other known steviol glycosides include rebaudioside B, C, D, E, F and M, steviolbioside and rubusoside.

Although methods are known for preparing steviol glycosides from Stevia rebaudiana, many of these methods are unsuitable for use commercially.

Accordingly, there remains a need for simple, efficient, and economical methods for preparing compositions comprising steviol glycosides, including highly purified steviol glycoside compositions.

### SUMMARY OF THE INVENTION

The present invention provides a process for preparing a composition comprising a target steviol glycoside by contacting a starting composition comprising an organic substrate with a microbial cell and/or enzyme preparation, thereby producing a composition comprising a target steviol glycoside.

The starting composition can be any organic compound comprising at least one carbon atom. In one embodiment, the starting composition is selected from the group consisting of steviol glycosides, polyols or sugar alcohols, various carbohydrates.

The target steviol glycoside can be any steviol glycoside. In one embodiment, the target steviol glycoside is steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, rebaudioside AM or a synthetic steviol glycoside.

In one embodiment, the target steviol glycoside is rebaudioside AM.

In some preferred embodiments enzyme preparation comprising one or more enzymes, or a microbial cell comprising one or more enzymes, capable of converting the starting composition to target steviol glycosides are used. The enzyme can be located on the surface and/or inside the cell. The enzyme preparation can be provided in the form of a whole cell suspension, a crude lysate or as purified enzyme(s). The enzyme preparation can be in free form or immobilized to a solid support made from inorganic or organic materials.

In some embodiments, a microbial cell comprises the necessary enzymes and genes encoding thereof for converting the starting composition to target steviol glycosides.

Accordingly, the present invention also provides a process for preparing a composition comprising a target steviol glycoside by contacting a starting composition comprising an organic substrate with a microbial cell comprising at least one enzyme capable of converting the starting composition to target steviol glycosides, thereby producing a medium comprising at least one target steviol glycoside.

The enzymes necessary for converting the starting composition to target steviol glycosides include the steviol biosynthesis enzymes, UDP-glucosyltransferases (UGTs) and/or UDP-recycling enzyme.

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

As used hereinafter, the term “SuSy_AT”, unless specified otherwise, refers to sucrose synthase having amino-acid sequence “SEQ ID 1” as described in Example 1.

As used hereinafter, the term “UGTS12”, unless specified otherwise, refers to UDP-glucosyltransferase having amino-acid sequence “SEQ ID 2” as described in Example 1.

As used hereinafter, the term “UGT76G1”, unless specified otherwise, refers to UDP-glucosyltransferase having amino-acid sequence “SEQ ID 3” as described in Example 1.

In one embodiment, steviol biosynthesis enzymes and UDP-glucosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2, UGTS12, EUGT11 and UGTs having substantial (>85%, >86%, >87%, >88%, >89%, >90%, >91%, >92%, >93%, >94%, >95%, >96%,>97%, >98%, >99%) amino-acid sequence identity to these polypeptides as well as isolated nucleic acid molecules that code for these UGTs.

In one embodiment, steviol biosynthesis enzymes, UGTs and UDP-glucose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the

UDP-glucosyltransferase is UGT85C2, or a UGT having >85% amino-acid sequence identity with UGT85C2.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with UGT74G1.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to the existing glucose at C13 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside to form steviolbioside.

In another embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside A (rebaudioside KA). In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside.

In another embodiment, the UDP-glucosyltransferase is any UDP-glucosyltransferase capable of adding at least one glucose unit to steviolbioside to form stevioside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside B to form rebaudioside E2.

In another embodiment, the UDP-glucosyltransferase is any UDP-glucosyltransferase capable of adding at least one glucose unit to stevioside A (rebaudioside KA) to form rebaudioside E3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A (rebaudioside KA) to form rebaudioside E

In another embodiment, the UDP-glucosyltransferase is any UDP-glucosyltransferase capable of adding at least one glucose unit to stevioside C to form rebaudioside E3. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to stevioside to form rebaudioside E2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside to form rebaudioside E. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E3 to form rebaudioside AM.

In another embodiment, the UDP-glucosyltransferase is any UDP-glucosyltransferase capable of adding at least one glucose unit to rebaudioside E2 to form rebaudioside AM. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E to form rebaudioside AM. In a particular embodiment, the UDP-glucosyltransferase is UGT76G1 or a UGT having >85% amino-acid sequence identity with UGT76G1.

Optionally, the method of the present invention further comprises using more than one UGT on a starting composition, to give a target steviol glycoside(s) having more than one glucose unit than the starting composition. In a particular embodiment, the UDP-glucosyltransferases are UGT74G1, UGT85C2, UGT76G1, UGTS12, EUGT11 and/or UGT91D2 or any UGT having >85% amino-acid sequence identity with UGT74G1, UGT85C2, UGT76G1, UGTS12, EUGT11 and/or UGT91D2 or any combination thereof, capable of adding more than one glucose unit to a starting composition to give a steviol glycoside(s) having more than one glucose unit than the starting composition.

In one embodiment, the UDP-glucosyltransferases are any UDP-glucosyltransferases capable of adding overall two glucose unit to stevioside to form rebaudioside AM. In a particular embodiment, the UDP-glucosyltransferases are selected from UGTS12, EUGT11, UGT91D2, UGT76G1 or any UGT having >85% amino-acid sequence identity with UGTS12, EUGT11, UGT91D2, UGT76G1 or any combination thereof. In another particular embodiment, the UDP-glucosyltransferases are UGTS12 and UGT76G1.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose.

In one embodiment, the recycling catalyst is sucrose synthase SuSy_At or a sucrose synthase having >85% amino-acid sequence identity with SuSy_At.

In one embodiment, the recycling substrate is sucrose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside AM.

Optionally, the method of the present invention further comprises separating the target steviol glycoside from the medium to provide a highly purified target steviol glycoside composition. The target steviol glycoside can be separated by at least one suitable method, such as, for example, crystallization, separation by membranes, centrifugation, extraction, chromatographic separation or a combination of such methods.

In one embodiment, the target steviol glycoside can be produced within the microorganism. In another embodiment, the target steviol glycoside can be secreted out in the medium. In one another embodiment, the released steviol glycoside can be continuously removed from the medium. In yet another embodiment, the target steviol glycoside is separated after the completion of the conversion reaction.

In one embodiment, separation produces a composition comprising greater than about 80% by weight of the target steviol glycoside on an anhydrous basis, i.e., a highly purified steviol glycoside composition. In another embodiment, separation produces a composition comprising greater than about 90% by weight of the target steviol glycoside. In particular embodiments, the composition comprises greater than about 95% by weight of the target steviol glycoside. In other embodiments, the composition comprises greater than about 99% by weight of the target steviol glycoside.

The target steviol glycoside can be in any polymorphic or amorphous form, including hydrates, solvates, anhydrous or combinations thereof.

Purified target steviol glycosides can be used in consumable products as a sweetener, flavor modifier, flavor with modifying properties and/or foaming suppressor. Suitable consumable products include, but are not limited to, food, beverages, pharmaceutical compositions, tobacco products, nutraceutical compositions, oral hygiene compositions, and cosmetic compositions.

### BRIEF DESCRIPTION OF THE DRAWINGS

FIG. 1 shows the chemical structure of rebaudioside AM.

FIG. 2 shows the pathways of producing rebaudioside AM and various steviol glycosides from steviol.

FIG. 3 shows the biocatalytic production of rebaudioside AM from stevioside using the enzymes UGTS12 and UGT76G1 and concomitant recycling of UDP to UDP-glucose via sucrose synthase SuSy_At.

FIG. 4 shows the biocatalytic production of rebausioside AM from rebaudioside E using the enzyme UGT76G1 and concomitant recycling of UDP to UDP-glucose via sucrose synthase SuSy_At.

FIG. 5 shows the HPLC chromatogram of stevioside. The peak with retention time of 25.992 minutes corresponds to stevioside.

FIG. 6 shows the HPLC chromatogram of the product of the biocatalytic production of rebaudioside AM from stevioside. The peak with retention time of 10.636 minutes corresponds to rebaudioside AM.

FIG. 7 shows the HPLC chromatogram of rebaudioside E. The peak with retention time of 10.835 minutes corresponds to rebaudioside E.

FIG. 8 shows the HPLC chromatogram of the product of the biocatalytic production of rebaudioside AM from rebaudioside E. The peaks with retention time of 10.936 and 11.442 minutes correspond to rebaudioside E and rebaudioside AM respectively.

FIG. 9 shows the HPLC chromatogram of rebaudioside AM after purification by methanol crystallization. The peak with retention time of 10.336 minutes corresponds to rebaudioside

FIG. 10 shows the ¹H NMR spectrum of rebaudioside AM (500 MHz, pyridine-d5).

FIG. 11 shows the HSQC spectrum of rebaudioside AM (500 MHz, pyridine-d5).

FIG. 12 shows the H,H COSY spectrum of rebaudioside AM (500 MHz, pyridine-d5).

FIG. 13 shows the HMBC spectrum of rebaudioside AM (500 MHz, pyridine-d5).

FIG. 14 shows the HSQC-TOCSY spectrum of rebaudioside AM (500 MHz, pyridine-d5).

FIG. 15a and FIG. 15b show the LC chromatogram and mass spectrum of rebaudioside AM respectively.

FIG. 16 is a graph showing the effect of Reb AM on the flavor modification of coconut water.

FIG. 17 is a graph showing the effect of Reb AM on the flavor modification of a chocolate protein shake.

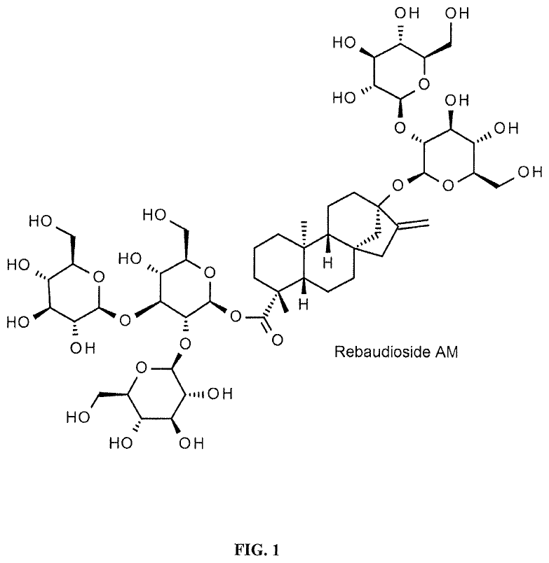

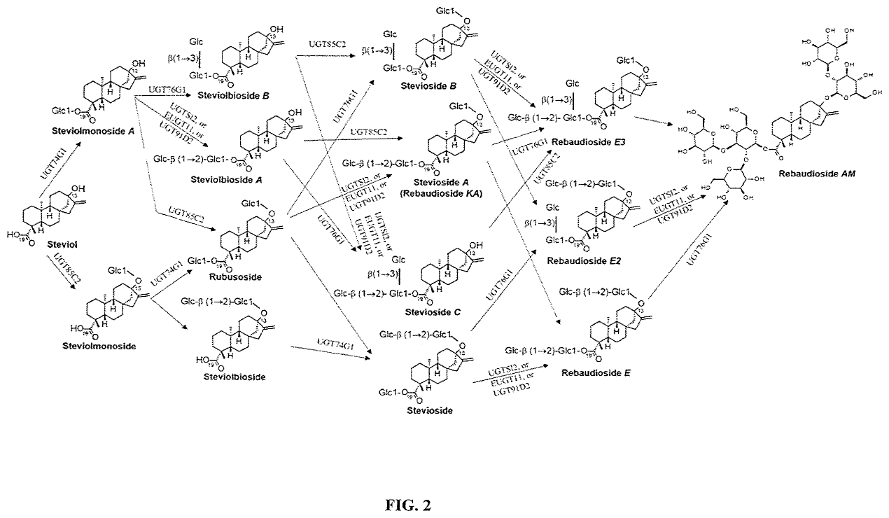

### DETAILED DESCRIPTION

The present invention provides a process for preparing a composition comprising a target steviol glycoside by contacting a starting composition comprising an organic substrate with a microbial cell and/or enzyme preparation, thereby producing a composition comprising a target steviol glycoside.

One object of the invention is to provide an efficient biocatalytic method for preparing target steviol glycosides, particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, rebaudioside AM or a synthetic steviol glycoside from various starting compositions.

As used herein, the abbreviation term “reb” refers to “rebaudioside”. Both terms have the same meaning and may be used interchangeably.

As used herein, “biocatalysis” or “biocatalytic” refers to the use of natural or genetically engineered biocatalysts, such as enzymes, or cells including microorganisms, comprising one or more enzyme, capable of single or multiple step chemical transformations on organic compounds. Biocatalysis processes include fermentation, biosynthesis, bioconversion and biotransformation processes. Both isolated enzyme, and whole-cell biocatalysis methods are known in the art. Biocatalyst protein enzymes can be naturally occurring or recombinant proteins.

As used herein, the term “steviol glycoside(s)” refers to a glycoside of steviol, including, but not limited to, naturally occurring steviol glycosides, e.g. steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, rebaudioside AM, synthetic steviol glycosides, e.g. enzymatically glucosylated steviol glycosides and combinations thereof.

### Starting Composition

As used herein, “starting composition” refers to any composition (generally an aqueous solution) containing one or more organic compound comprising at least one carbon atom.

In one embodiment, the starting composition is selected from the group consisting of steviol, steviol glycosides, polyols and various carbohydrates.

The starting composition steviol glycoside is selected from the group consisting of steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 or other glycoside of steviol occurring in Stevia rebaudiana plant, synthetic steviol glycosides, e.g. enzymatically glucosylated steviol glycosides and combinations thereof.

In one embodiment, the starting composition is steviol.

In another embodiment, the starting composition steviol glycoside is steviolmonoside.

In yet another embodiment, the starting composition steviol glycoside is steviolmonoside A.

In still another embodiment, the starting composition steviol glycoside is rubusoside.

In yet another embodiment, the starting composition steviol glycoside is steviolbioside.

In yet another embodiment, the starting composition steviol glycoside is steviolbioside A.

In yet another embodiment, the starting composition steviol glycoside is steviolbioside B.

In still another embodiment, the starting composition steviol glycoside is stevioside.

In yet another embodiment, the starting composition steviol glycoside is stevioside A, also known as rebaudioside KA.

In still another embodiment, the starting composition steviol glycoside is stevioside B.

In still another embodiment, the starting composition steviol glycoside is stevioside C.

In another embodiment, the starting composition steviol glycoside is rebaudioside E.

In another embodiment, the starting composition steviol glycoside is rebaudioside E2.

In another embodiment, the starting composition steviol glycoside is rebaudioside E3.

The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced.

The term “carbohydrate” refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

The starting composition may be synthetic or purified (partially or entirely), commercially available or prepared.

In one embodiment, the starting composition is glycerol.

In another embodiment, the starting composition is glucose.

In still another embodiment, the starting composition is sucrose.

In yet another embodiment, the starting composition is starch.

In another embodiment, the starting composition is maltodextrin.

In yet another embodiment, the starting composition is cellulose.

In still another embodiment, the starting composition is amylose.

The organic compound(s) of starting composition serve as a substrate(s) for the production of the target steviol glycoside(s), as described herein.

### Target Steviol Glycoside

The target steviol glycoside of the present method can be any steviol glycoside that can be prepared by the process disclosed herein. In one embodiment, the target steviol glycoside is selected from the group consisting of steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, rebaudioside AM or other glycoside of steviol occurring in Stevia rebaudiana plant, synthetic steviol glycosides, e.g. enzymatically glucosylated steviol glycosides and combinations thereof.

In one embodiment, the target steviol glycoside is steviolmonoside.

In another embodiment, the target steviol glycoside is steviolmonoside A.

In another embodiment, the target steviol glycoside is steviolbioside.

In another embodiment, the target steviol glycoside is steviolbioside A.

In another embodiment, the target steviol glycoside is steviolbioside B.

In another embodiment, the target steviol glycoside is rubusoside.

In another embodiment, the target steviol glycoside is stevioside.

In another embodiment, the target steviol glycoside is stevioside A (rebaudioside KA).

In another embodiment, the target steviol glycoside is stevioside B.

In another embodiment, the target steviol glycoside is stevioside C.

In another embodiment, the target steviol glycoside is rebaudioside E.

In another embodiment, the target steviol glycoside is rebaudioside E2.

In another embodiment, the target steviol glycoside is rebaudioside E3.

In another embodiment, the target steviol glycoside is rebaudioside AM.

The target steviol glycoside can be in any polymorphic or amorphous form, including hydrates, solvates, anhydrous or combinations thereof.

In one embodiment, the present invention is a biocatalytic process for the production of steviolmonoside.

In one embodiment, the present invention is a biocatalytic process for the production of steviolmonoside A.

In one embodiment, the present invention is a biocatalytic process for the production of steviolbioside.

In one embodiment, the present invention is a biocatalytic process for the production of steviolbioside A.

In one embodiment, the present invention is a biocatalytic process for the production of steviolbioside B.

In one embodiment, the present invention is a biocatalytic process for the production of rubusoside.

In one embodiment, the present invention is a biocatalytic process for the production of stevioside.

In one embodiment, the present invention is a biocatalytic process for the production of stevioside A (rebaudioside KA).

In one embodiment, the present invention is a biocatalytic process for the production of stevioside B.

In one embodiment, the present invention is a biocatalytic process for the production of stevioside C.

In one embodiment, the present invention is a biocatalytic process for the production of rebaudioside E.

In one embodiment, the present invention is a biocatalytic process for the production of rebaudioside E2.

In one embodiment, the present invention is a biocatalytic process for the production of rebaudioside E3.

In one embodiment, the present invention is a biocatalytic process for the production of rebaudioside AM.

In a particular embodiment, the present invention provides for the biocatalytic process for the production of rebaudioside AM from a starting composition comprising stevioside and UDP-glucose.

In another particular embodiment, the present invention provides for the biocatalytic process for the production of rebaudioside AM from a starting composition comprising rebaudioside E and UDP-glucose.

Optionally, the method of the present invention further comprises separating the target steviol glycoside from the medium to provide a highly purified target steviol glycoside composition. The target steviol glycoside can be separated by any suitable method, such as, for example, crystallization, separation by membranes, centrifugation, extraction, chromatographic separation or a combination of such methods.

In particular embodiments, the process described herein results in a highly purified target steviol glycoside composition. The term “highly purified”, as used herein, refers to a composition having greater than about 80% by weight of the target steviol glycoside on an anhydrous (dried) basis. In one embodiment, the highly purified target steviol glycoside composition contains greater than about 90% by weight of the target steviol glycoside on an anhydrous (dried) basis, such as, for example, greater than about 91%, greater than about 92%, greater than about 93%, greater than about 94%, greater than about 95%, greater than about 96%, greater than about 97%, greater than about 98% or greater than about 99% target steviol glycoside content on a dried basis.

In one embodiment, when the target steviol glycoside is reb AM, the process described herein provides a composition having greater than about 90% reb AM content by weight on a dried basis. In another particular embodiment, when the target steviol glycoside is reb AM, the process described herein provides a composition comprising greater than about 95% reb AM content by weight on a dried basis.

### Microorganisms and Enzyme Preparations

In one embodiment of present invention, a microorganism (microbial cell) and/or enzyme preparation is contacted with a medium containing the starting composition to produce target steviol glycosides.

The enzyme can be provided in the form of a whole cell suspension, a crude lysate, a purified enzyme or a combination thereof. In one embodiment, the biocatalyst is a purified enzyme capable of converting the starting composition to the target steviol glycoside. In another embodiment, the biocatalyst is a crude lysate comprising at least one enzyme capable of converting the starting composition to the target steviol glycoside. In still another embodiment, the biocatalyst is a whole cell suspension comprising at least one enzyme capable of converting the starting composition to the target steviol glycoside.

In another embodiment, the biocatalyst is one or more microbial cells comprising enzyme(s) capable of converting the starting composition to the target steviol glycoside. The enzyme can be located on the surface of the cell, inside the cell or located both on the surface of the cell and inside the cell.

Suitable enzymes for converting the starting composition to target steviol glycosides include, but are not limited to, the steviol biosynthesis enzymes and UDP-glucosyltransferases (UGTs). Optionally it may include UDP recycling enzyme(s).

In one embodiment, the steviol biosynthesis enzymes include mevalonate (MVA) pathway enzymes.

In another embodiment, the steviol biosynthesis enzymes include non-mevalonate 2-C-methyl-D-erythritol-4-phosphate pathway (MEP/DOXP) enzymes.

In one embodiment the steviol biosynthesis enzymes are selected from the group including geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase etc.

The UDP-glucosyltransferase can be any UDP-glucosyltransferase capable of adding at least one glucose unit to steviol and/or a steviol glycoside substrate to provide the target steviol glycoside.

In one embodiment, steviol biosynthesis enzymes and UDP-glucosyltransferases are produced in a microbial cell. The microbial cell may be, for example, E. coli, Saccharomyces sp., Aspergillus sp., Pichia sp., Bacillus sp., Yarrowia sp. etc. In another embodiment, the UDP-glucosyltransferases are synthesized.

In one embodiment, the UDP-glucosyltransferase is selected from group including UGT74G1, UGT85C2, UGT76G1, UGT91D2, UGTS12, EUGT11 and UGTs having substantial (>85%, >86%, >87%, >88%, >89%, >90%, >91%, >92%, >93%, >94%, >95%, >96%,>97%, >98%, >99%) amino-acid sequence identity to these polypeptides as well as isolated nucleic acid molecules that code for these UGTs.

In one embodiment, steviol biosynthesis enzymes, UGTs and UDP-glucose recycling system are present in one microorganism (microbial cell). The microorganism may be for example, E. coli, Saccharomyces sp., Aspergillus sp., Pichia sp., Bacillus sp., Yarrowia sp.

In one embodiment, the UDP-glucosyltransferase is any UDP-glucosyltransferase capable of adding at least one glucose unit to steviol or any starting steviol glycoside bearing an —OH functional group at C13 to give a target steviol glycoside having an —O-glucose beta glucopyranoside glycosidic linkage at C13. In a particular embodiment, the UDP-glucosyltransferase is UGT85C2, or a UGT having >85% amino-acid sequence identity with UGT85C2.

In another embodiment, the UDP-glucosyltransferase is any UDP-glucosyltransferase capable of adding at least one glucose unit to steviol or any starting steviol glycoside bearing a —COOH functional group at C19 to give a target steviol glycoside having a —COO-glucose beta-glucopyranoside glycosidic linkage at C19. In a particular embodiment, the UDP-glucosyltransferase is UGT74G1, or a UGT having >85% amino-acid sequence identity with UGT74G1.

In another embodiment, the UDP-glucosyltransferase is any UDP-glucosyltransferase capable of adding at least one glucose unit to the existing glucose at C19 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to the existing glucose at

C19 of any starting steviol glycoside to give a target steviol glycoside with at least one additional glucose bearing at least one beta 1→3 glucopyranoside glycosidic linkage(s) at the newly formed bond glycosidic bond(s). In a particular embodiment, the UDP-glucosyltransferase is UGT76G1, or a UGT having >85% amino-acid sequence identity with UGT76G1.

In another embodiment, the UDP-glucosyltransferase is any UDP-glucosyltransferase capable of adding at least one glucose unit to the existing glucose at C13 of any starting steviol glycoside to give a target steviol glycoside with at least one additional glucose bearing at least one beta 1→2 glucopyranoside glycosidic linkage(s) at the newly formed glycosidic bond(s). In a particular embodiment, the UDP-glucosyltransferase is UGTS12, or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In one embodiment, the UDP-glucosyltransferase is any UDP-glucosyltransferase capable of adding at least one glucose unit to steviol to form steviolmonoside. In a particular embodiment, the UDP-glucosyltransferase is UGT85C2 or a UGT having >85% amino-acid sequence identity with UGT85C2 or a UGT having >85% amino-acid sequence identity with UGT85C2.

In another embodiment, the UDP-glucosyltransferase is any UDP-glucosyltransferase capable of adding at least one glucose unit to steviol to form steviolmonoside A. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside A to form steviolbioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to steviolmonoside A to form steviolbioside A. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolmonoside A to form rubusoside. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolmonoside to form rubusoside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to steviolmonoside to form steviolbioside.

In another embodiment, the UDP-glucosyltransferase is any UDP-glucosyltransferase capable of adding at least one glucose unit to steviolbioside B to form stevioside B. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolbioside B to form stevioside C. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to steviolbioside A to form stevioside A. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to steviolbioside A to form stevioside C. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside B. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to rubusoside to form stevioside A (rebaudioside KA). In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rubusoside to form stevioside.

In another embodiment, the UDP-glucosyltransferase is any UDP-glucosyltransferase capable of adding at least one glucose unit to steviolbioside to form stevioside. In a particular embodiment, the UDP-glucosyltransferase is UGT74G1 or a UGT having >85% amino-acid sequence identity with UGT74G1.

In another embodiment, the UDP-glucosyltransferase is any UDP-glucosyltransferase capable of adding at least one glucose unit to stevioside B to form rebaudioside E3. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to stevioside B to form rebaudioside E2.

In another embodiment, the UDP-glucosyltransferase is any UDP-glucosyltransferase capable of adding at least one glucose unit to stevioside A (rebaudioside KA) to form rebaudioside E3. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside A (rebaudioside KA) to form rebaudioside E

In another embodiment, the UDP-glucosyltransferase is any UDP-glucosyltransferase capable of adding at least one glucose unit to stevioside C to form rebaudioside E3. In a particular embodiment, the UDP-glucosyltransferase is UGT85C2 or a UGT having >85% amino-acid sequence identity with UGT85C2.

In another embodiment, the UDP-glucosyltransferase is any UDP-glucosyltransferase capable of adding at least one glucose unit to stevioside to form rebaudioside E2. In a particular embodiment, the UDP-glucosyltransferase is UGT76G1 or a UGT having >85% amino-acid sequence identity with UGT76G1.

In another embodiment, the UDP-glucosyltransferase is any UDP-glucosyltransferase capable of adding at least one glucose unit to stevioside to form rebaudioside E. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E3 to form rebaudioside AM.

In another embodiment, the UDP-glucosyltransferase is any UDP-glucosyltransferase capable of adding at least one glucose unit to rebaudioside E2 to form rebaudioside AM. In a particular embodiment, the UDP-glucosyltransferase is UGTS12 or a UGT having >85% amino-acid sequence identity with UGTS12. In another particular embodiment, the UDP-glucosyltransferase is EUGT11, or a UGT having >85% amino-acid sequence identity with EUGT11. In yet another particular embodiment, the UDP-glucosyltransferase is UGT91D2, or a UGT having >85% amino-acid sequence identity with UGT91D2.

In another embodiment, the UDP-glucosyltransferase is any UDP-glucosyltransferase capable of adding at least one glucose unit to rebaudioside E to form rebaudioside AM. In a particular embodiment, the UDP-glucosyltransferase is UGT76G1 or a UGT having >85% amino-acid sequence identity with UGT76G1.

Optionally, the method of the present invention further comprises using more than one UGT on a starting composition, to give a target steviol glycoside(s) having more than one glucose unit than the starting composition. In a particular embodiment, the UDP-glucosyltransferases are UGT74G1, UGT85C2, UGT76G1, UGTS12, EUGT11 and/or UGT91D2 or any UGT having >85% amino-acid sequence identity with UGT74G1, UGT85C2, UGT76G1, UGTS12, EUGT11 and/or UGT91D2 or any combination thereof, capable of adding more than one glucose unit to a starting composition to give a steviol glycoside(s) having more than one glucose unit than the starting composition.

In one embodiment, the UDP-glucosyltransferases are any UDP-glucosyltransferases capable of adding overall two glucose unit to stevioside to form rebaudioside AM. In a particular embodiment, the UDP-glucosyltransferases are selected from UGTS12, EUGT11, UGT91D2, UGT76G1 or any UGT having >85% amino-acid sequence identity with UGTS12, EUGT11, UGT91D2, UGT76G1 or any combination thereof. In another particular embodiment, the UDP-glucosyltransferases are UGTS12 and UGT76G1.

Optionally, the method of the present invention further comprises recycling UDP to provide UDP-glucose. In one embodiment, the method comprises recycling UDP by providing a recycling catalyst and a recycling substrate, such that the biotransformation of steviol and/or the steviol glycoside substrate to the target steviol glycoside is carried out using catalytic amounts of UDP-glucosyltransferase and UDP-glucose. The UDP recycling enzyme can be sucrose synthase SuSy_At or a sucrose synthase having >85% amino-acid sequence identity with SuSy_At and the recycling substrate can be sucrose.

Optionally, the method of the present invention further comprises the use of transglycosidases that use oligo- or poly-saccharides as the sugar donor to modify recipient target steviol glycoside molecules. Non-limiting examples include cyclodextrin glycosyltransferase (CGTase), fructofuranosidase, amylase, saccharase, glucosucrase, beta-h-fructosidase, beta.-fructosidase, sucrase, fructosylinvertase, alkaline invertase, acid invertase, fructofuranosidase. In some embodiments, glucose and sugar(s) other than glucose, including but not limited to fructose, xylose, rhamnose, arabinose, deoxyglucose, galactose are transferred to the recipient target steviol glycosides. In one embodiment, the recipient steviol glycoside is rebaudioside AM.

In another embodiment, the UDP-glucosyltransferase capable of adding at least one glucose unit to starting composition steviol glycoside has >85% amino-acid sequence identity with UGTs selected from the following listing of GenInfo identifier numbers, preferably from the group presented in Table 1, and Table 2.

| | | | | | |
|---|---|---|---|---|---|
| 397567 | 30680413 | 115480946 | 147798902 | 218193594 | 225443294 |
| 454245 | 32816174 | 116310259 | 147811764 | 218193942 | 225444853 |
| 1359905 | 32816178 | 116310985 | 147827151 | 219885307 | 225449296 |
| 1685003 | 34393978 | 116788066 | 147836230 | 222615927 | 225449700 |
| 1685005 | 37993665 | 116788606 | 147839909 | 222619587 | 225454338 |
| 2191136 | 37993671 | 116789315 | 147846163 | 222623142 | 225454340 |
| 2501497 | 37993675 | 119394507 | 147855977 | 222625633 | 225454342 |
| 2911049 | 39104603 | 119640480 | 148905778 | 222625635 | 225454473 |
| 4218003 | 41469414 | 122209731 | 148905999 | 222636620 | 225454475 |
| 4314356 | 41469452 | 125526997 | 148906835 | 222636621 | 225458362 |
| 13492674 | 42566366 | 125534279 | 148907340 | 222636628 | 225461551 |
| 13492676 | 42570280 | 125534461 | 148908935 | 222636629 | 225461556 |
| 15217773 | 42572855 | 125540090 | 148909182 | 224053242 | 225461558 |
| 15217796 | 44890129 | 125541516 | 148909920 | 224053386 | 225469538 |
| 15223396 | 46806235 | 125545408 | 148910082 | 224055535 | 225469540 |
| 15223589 | 50284482 | 125547340 | 148910154 | 224056138 | 226316457 |
| 15227766 | 51090402 | 125547520 | 148910612 | 224056160 | 226492603 |
| 15230017 | 51090594 | 125554547 | 148910769 | 224067918 | 226494221 |
| 15231757 | 52839682 | 125557592 | 156138791 | 224072747 | 226495389 |
| 15234056 | 56550539 | 125557593 | 156138797 | 224080189 | 226495945 |
| 15234195 | 62734263 | 125557608 | 156138799 | 224091845 | 226502400 |
| 15234196 | 62857204 | 125559566 | 156138803 | 224094703 | 226507980 |
| 15238503 | 62857206 | 125563266 | 165972256 | 224100653 | 226531147 |
| 15239523 | 62857210 | 125571055 | 168016721 | 224100657 | 226532094 |
| 15239525 | 62857212 | 125579728 | 171674071 | 224101569 | 238477377 |
| 15239543 | 75265643 | 125588307 | 171906258 | 224103105 | 240254512 |
| 15239937 | 75285934 | 125589492 | 183013901 | 224103633 | 242032615 |
| 15240305 | 75288884 | 125599469 | 183013903 | 224103637 | 242032621 |
| 15240534 | 77550661 | 125601477 | 186478321 | 224109218 | 242038423 |
| 15982889 | 77556148 | 126635837 | 187373030 | 224114583 | 242043290 |
| 18086351 | 82791223 | 126635845 | 187373042 | 224116284 | 242044836 |
| 18418378 | 83778990 | 126635847 | 190692175 | 224120552 | 242051252 |
| 18418380 | 89953335 | 126635863 | 194701936 | 224121288 | 242056217 |
| 18418382 | 110741436 | 126635867 | 195620060 | 224121296 | 242056219 |
| 19743740 | 110743955 | 126635883 | 209954691 | 224121300 | 242056663 |
| 19911201 | 115438196 | 126635887 | 209954719 | 224130358 | 242059339 |
| 20149064 | 115438785 | 133874210 | 209954725 | 224140703 | 242059341 |
| 20260654 | 115441237 | 133874212 | 209954733 | 224143404 | 242060922 |
| 21435782 | 115454819 | 145358033 | 210063105 | 224143406 | 242067411 |
| 21553613 | 115456047 | 147772508 | 210063107 | 224144306 | 242067413 |
| 21593514 | 115457492 | 147776893 | 212275846 | 224285244 | 242076258 |
| 22759895 | 115459312 | 147776894 | 216296854 | 225431707 | 242076396 |
| 23955910 | 115464719 | 147776895 | 217074506 | 225435532 | 242084750 |
| 26452040 | 115471069 | 147786916 | 218185693 | 225436321 | 242091005 |
| 28393204 | 115471071 | 147798900 | 218187075 | 225440041 | 242095206 |
| 30679796 | 115474009 | 147798901 | 218189427 | 225441116 | 242345159 |
| 242345161 | 297724601 | 326492035 | 356523945 | 357140904 | 359486938 |
| 255536859 | 297725463 | 326493430 | 356523957 | 357165849 | 359487055 |
| 255538228 | 297728331 | 326500410 | 356523959 | 357165852 | 359488135 |
| 255541676 | 297738632 | 326506816 | 356523961 | 357168415 | 359488708 |
| 255547075 | 297745347 | 326507826 | 356523963 | 357437837 | 359493630 |
| 255552620 | 297745348 | 326508394 | 356524387 | 357442755 | 359493632 |
| 255552622 | 297795735 | 326509445 | 356524403 | 357442757 | 359493634 |
| 255555343 | 297796253 | 326511261 | 356527181 | 357445729 | 359493636 |
| 255555361 | 297796257 | 326511866 | 356533209 | 357445731 | 359493815 |
| 255555363 | 297796261 | 326512412 | 356533852 | 357445733 | 359495856 |
| 255555365 | 297797587 | 326517673 | 356534718 | 357446799 | 359495858 |
| 255555369 | 297798502 | 326518800 | 356535480 | 357446805 | 359495869 |
| 255555373 | 297799226 | 326521124 | 356542996 | 357452779 | 359495871 |
| 255555377 | 297805988 | 326525567 | 356543136 | 357452781 | 359497638 |
| 255556812 | 297807499 | 326525957 | 356543932 | 357452783 | 359807261 |
| 255556818 | 297809125 | 326526607 | 356549841 | 357452787 | 374256637 |
| 255563008 | 297809127 | 326527141 | 356549843 | 357452789 | 377655465 |
| 255564074 | 297811403 | 326530093 | 356554358 | 357452791 | 378405177 |
| 255564531 | 297820040 | 326534036 | 356554360 | 357452797 | 378829085 |
| 255572878 | 297821483 | 326534312 | 356558606 | 357452799 | 387135070 |
| 255577901 | 297825217 | 332071132 | 356560333 | 357470367 | 387135072 |
| 255583249 | 297832276 | 339715876 | 356560599 | 357472193 | 387135078 |
| 255583253 | 297832280 | 342306012 | 356560749 | 357472195 | 387135092 |
| 255583255 | 297832518 | 342306016 | 356566018 | 357474295 | 387135094 |
| 255585664 | 297832520 | 343457675 | 356566169 | 357474493 | 387135098 |
| 255585666 | 297840825 | 343457677 | 356566173 | 357474497 | 387135100 |
| 255634688 | 297840827 | 350534960 | 356567761 | 357474499 | 387135134 |
| 255644801 | 297847402 | 356498085 | 356574704 | 357490035 | 387135136 |
| 255645821 | 297849372 | 356499771 | 356576401 | 357493567 | 387135174 |
| 255647456 | 300078590 | 356499777 | 356577660 | 357497139 | 387135176 |
| 255648275 | 300669727 | 356499779 | 357114993 | 357497581 | 387135184 |
| 260279126 | 302142947 | 356501328 | 357115447 | 357497671 | 387135186 |
| 260279128 | 302142948 | 356502523 | 357115451 | 357500579 | 387135188 |
| 261343326 | 302142950 | 356503180 | 357115453 | 357504663 | 387135190 |
| 283132367 | 302142951 | 356503184 | 357116080 | 357504691 | 387135192 |
| 283362112 | 302765302 | 356503295 | 357116928 | 357504699 | 387135194 |
| 289188052 | 302796334 | 356504436 | 357117461 | 357504707 | 387135282 |
| 295841350 | 302811470 | 356504523 | 357117463 | 357505859 | 387135284 |
| 296088529 | 302821107 | 356504765 | 357117829 | 357510851 | 387135294 |
| 296090415 | 302821679 | 356511113 | 357117839 | 357516975 | 387135298 |
| 296090524 | 319759260 | 356515120 | 357125059 | 359477003 | 387135300 |
| 296090526 | 319759266 | 356517088 | 357126015 | 359477998 | 387135302 |
| 297599503 | 320148814 | 356520732 | 357134488 | 359478043 | 387135304 |
| 297601531 | 326489963 | 356522586 | 357135657 | 359478286 | 387135312 |
| 297611791 | 326490273 | 356522588 | 357138503 | 359484299 | 387135314 |
| 297722841 | 326491131 | 356522590 | 357139683 | 359486936 | 387135316 |
| 387135318 | 449440433 | 460376293 | 460413408 | 462423864 | 475546199 |
| 387135320 | 449445896 | 460378310 | 460416351 | 470101924 | 475556485 |
| 387135322 | 449446454 | 460380744 | 462394387 | 470102280 | 475559699 |
| 387135324 | 449447657 | 460381726 | 462394433 | 470102858 | 475578293 |
| 387135326 | 449449002 | 460382093 | 462394557 | 470104211 | 475591753 |
| 387135328 | 449449004 | 460382095 | 462395646 | 470104264 | 475593742 |
| 388493506 | 449449006 | 460382754 | 462395678 | 470104266 | 475612072 |
| 388495496 | 449451379 | 460384935 | 462396388 | 470106317 | 475622476 |
| 388498446 | 449451589 | 460384937 | 462396389 | 470106357 | 475622507 |
| 388499220 | 449451591 | 460385076 | 462396419 | 470115448 | 475623787 |
| 388502176 | 449451593 | 460385872 | 462396542 | 470130404 | 482550481 |
| 388517521 | 449453712 | 460386018 | 462397507 | 470131550 | 482550499 |
| 388519407 | 449453714 | 460389217 | 462399998 | 470136482 | 482550740 |
| 388521413 | 449453716 | 460394872 | 462400798 | 470136484 | 482550999 |
| 388827901 | 449453732 | 460396139 | 462401217 | 470136488 | 482552352 |
| 388827903 | 449457075 | 460397862 | 462402118 | 470136492 | 482554970 |
| 388827907 | 449467555 | 460397864 | 462402237 | 470137933 | 482555336 |
| 388827909 | 449468742 | 460398541 | 462402284 | 470137937 | 482555478 |
| 388827913 | 449495638 | 460403139 | 462402416 | 470140422 | 482556454 |
| 393887637 | 449495736 | 460403141 | 462404228 | 470140426 | 482557289 |
| 393887646 | 449499880 | 460403143 | 462406358 | 470140908 | 482558462 |
| 393887649 | 449502786 | 460403145 | 462408262 | 470141232 | 482558508 |
| 393990627 | 449503471 | 460405998 | 462409325 | 470142008 | 482558547 |
| 397746860 | 449503473 | 460407578 | 462409359 | 470142010 | 482561055 |
| 397789318 | 449515857 | 460407590 | 462409777 | 470142012 | 482561555 |
| 413924864 | 449518643 | 460409128 | 462411467 | 470143607 | 482562795 |
| 414590349 | 449519559 | 460409134 | 462414311 | 470143939 | 482562850 |
| 414590661 | 449522783 | 460409136 | 462414416 | 470145404 | 482565074 |
| 414591157 | 449524530 | 460409459 | 462414476 | 473923244 | 482566269 |
| 414879558 | 449524591 | 460409461 | 462415526 | 474114354 | 482566296 |
| 414879559 | 449528823 | 460409463 | 462415603 | 474143634 | 482566307 |
| 414879560 | 449528825 | 460409465 | 462415731 | 474202268 | 482568689 |
| 414888074 | 449534021 | 460409467 | 462416307 | 474299266 | 482570049 |
| 431812559 | 460365546 | 460410124 | 462416920 | 474363119 | 482570572 |
| 449432064 | 460366882 | 460410126 | 462416922 | 474366157 | 482575121 |
| 449432066 | 460369823 | 460410128 | 462416923 | 474429346 | |
| 449433069 | 460369829 | 460410130 | 462416924 | 475432777 | |
| 449436944 | 460369831 | 460410132 | 462417401 | 475473002 | |
| 449438665 | 460369833 | 460410134 | 462419769 | 475489790 | |
| 449438667 | 460370755 | 460410213 | 462420317 | 475511330 | |
| 449440431 | 460374714 | 460411200 | 462423366 | 475516200 | |

TABLE 1

| GI number | Accession | Origin |
|---|---|---|
| 190692175 | ACE87855.1 | Stevia rebaudiana |
| 41469452 | AAS07253.1 | Oryza saliva |
| 62857204 | BAD95881.1 | Ipomoea nil |
| 62857206 | BAD95882.1 | Ipomoea purperea |
| 56550539 | BAD77944.1 | Bellis perennis |
| 115454819 | NP_001051010.1 | Oryza sativa Japonica Group |
| 115459312 | NP_001053256.1 | Oryza sativa Japonica Group |
| 115471069 | NP_001059133.1 | Oryza saliva Japonica Group |
| 115471071 | NP_001059134.1 | Oryza saliva Japonica Group |
| 116310985 | CAH67920.1 | Oryza sativa Indica Group |
| 116788066 | ABK24743.1 | Picea sitchensis |
| 122209731 | Q2V6J9.1 | Fragaria × ananassa |
| 125534461 | EAY81009.1 | Oryza sativa Indica Group |
| 125559566 | EAZ05102.1 | Oryza sativa Indica Group |
| 125588307 | EAZ28971.1 | Oryza sativa Japonica Group |
| 148907340 | ABR16806.1 | Picea sitchensis |
| 148910082 | ABR18123.1 | Picea sitchensis |
| 148910612 | ABR18376.1 | Picea sitchensis |
| 15234195 | NP_194486.1 | Arabidopsis thaliana |
| 15239523 | NP_200210.1 | Arabidopsis thaliana |
| 15239937 | NP_196793.1 | Arabidopsis thaliana |
| 1685005 | AAB36653.1 | Nicotiana tabacum |
| 183013903 | ACC38471.1 | Medicago truncatula |
| 186478321 | NP_172511.3 | Arabidopsis thaliana |
| 187373030 | ACD03249.1 | Avena strigosa |
| 194701936 | ACF85052.1 | Zea mays |
| 19743740 | AAL92461.1 | Solanum lycopersicum |
| 212275846 | NP_001131009.1 | Zea mays |
| 222619587 | EEE55719.1 | Oryza sativa Japonica Group |
| 224055535 | XP_002298527.1 | Populus trichocarpa |
| 224101569 | XP_002334266.1 | Populus trichocarpa |
| 224120552 | XP_002318358.1 | Populus trichocarpa |
| 224121288 | XP_002330790.1 | Populus trichocarpa |
| 225444853 | XP_002281094 | Vitis vinifera |
| 225454342 | XP_002275850.1 | Vitis vinifera |
| 225454475 | XP_002280923.1 | Vitis vinifera |
| 225461556 | XP_002285222 | Vitis vinifera |
| 225469540 | XP_002270294.1 | Vitis vinifera |
| 226495389 | NP_001148083.1 | Zea mays |
| 226502400 | NP_001147674.1 | Zea mays |
| 238477377 | ACR43489.1 | Triticum aestivum |
| 240254512 | NP_565540.4 | Arabidopsis thaliana |
| 2501497 | Q43716.1 | Petunia × hybrida |
| 255555369 | XP_002518721.1 | Ricinus communis |
| 26452040 | BAC43110.1 | Arabidopsis thaliana |
| 296088529 | CBI37520.3 | Vitis vinifera |
| 297611791 | NP_001067852.2 | Oryza sativa Japonica Group |
| 297795735 | XP_002865752.1 | Arabidopsis lyrata subsp. lyrata |
| 297798502 | XP_002867135.1 | Arabidopsis lyrata subsp. lyrata |
| 297820040 | XP_002877903.1 | Arabidopsis lyrata subsp. lyrata |
| 297832276 | XP_002884020.1 | Arabidopsis lyrata subsp. lyrata |
| 302821107 | XP_002992218.1 | Selaginella moellendorffii |
| 30680413 | NP_179446.2 | Arabidopsis thaliana |
| 319759266 | ADV71369.1 | Pueraria montana var. lobata |
| 326507826 | BAJ86656.1 | Hordeum vulgare subsp. Vulgare |
| 343457675 | AEM37036.1 | Brassica rapa subsp. oleifera |
| 350534960 | NP_001234680.1 | Solanum lycopersicum |
| 356501328 | XP_003519477.1 | Glycine max |
| 356522586 | XP_003529927.1 | Glycine max |
| 356535480 | XP_003536273.1 | Glycine max |
| 357445733 | XP_003593144.1 | Medicago truncatula |
| 357452783 | XP_003596668.1 | Medicago truncatula |
| 357474493 | XP_003607531.1 | Medicago truncatula |
| 357500579 | XP_003620578.1 | Medicago truncatula |
| 357504691 | XP_003622634.1 | Medicago truncatula |
| 359477998 | XP_003632051.1 | Vitis vinifera |
| 359487055 | XP_002271587 | Vitis vinifera |
| 359495869 | XP_003635104.1 | Vitis vinifera |
| 387135134 | AFJ52948.1 | Linum usitatissimum |
| 387135176 | AFJ52969.1 | Linum usitatissimum |
| 387135192 | AFJ52977.1 | Linum usitatissimum |
| 387135282 | AFJ53022.1 | Linum usitatissimum |
| 387135302 | AFJ53032.1 | Linum usitatissimum |
| 387135312 | AFJ53037.1 | Linum usitatissimum |
| 388519407 | AFK47765.1 | Medicago truncatula |
| 393887646 | AFN26668.1 | Barbarea vulgaris subsp. arcuata |
| 414888074 | DAA64088.1 | Zea mays |
| 42572855 | NP_974524.1 | Arabidopsis thaliana |
| 449440433 | XP_004137989.1 | Cucumis sativus |
| 449446454 | XP_004140986.1 | Cucumis sativus |
| 449449004 | XP_004142255.1 | Cucumis sativus |
| 449451593 | XP_004143546.1 | Cucumis sativus |
| 449515857 | XP_004164964.1 | Cucumis sativus |
| 460382095 | XP_004236775.1 | Solanum lycopersicum |
| 460409128 | XP_004249992.1 | Solanum lycopersicum |
| 460409461 | XP_004250157.1 | Solanum lycopersicum |
| 460409465 | XP_004250159.1 | Solanum lycopersicum |
| 462396388 | EMJ02187.1 | Prunus persica |
| 462402118 | EMJ07675.1 | Prunus persica |
| 462409359 | EMJ14693.1 | Prunus persica |
| 462416923 | EMJ21660.1 | Prunus persica |
| 46806235 | BAD17459.1 | Oryza saliva Japonica Group |
| 470104266 | XP_004288529.1 | Fragaria vesca subsp. vesca |
| 470142008 | XP_004306714.1 | Fragaria vesca subsp. vesca |
| 475432777 | EMT01232.1 | Aegilops tauschii |
| 51090402 | BAD35324.1 | Oryza sativa Japonica Group |

TABLE 2

| | | | Internal |
|---|---|---|---|
| GI number | Accession | Origin | reference |
| 460409128 | XP.004249992.1 | Solanum lycopersicum | UGTSl |
| 460386018 | XP.004238697.1 | Solanum lycopersicum | — |
| 460409134 | XP.004249995.1 | Solanum lycopersicum | — |
| 460410132 | XP.004250485.1 | Solanum lycopersicum | UGTSl2 |
| 460410130 | XP.004250484.1 | Solanum lycopersicum | — |
| 460410128 | XP.004250483.1 | Solanum lycopersicum | — |
| 460378310 | XP.004234916.1 | Solanum lycopersicum | — |
| 209954733 | BAG80557.1 | Lycium barbarum | UGTLB |
| 209954725 | BAG80553.1 | Lycium barbarum | — |

One embodiment of the present invention is a microbial cell comprising an enzyme, i.e. an enzyme capable of converting the starting composition to the target steviol glycoside. Accordingly, some embodiments of the present method include contacting a microorganism with a medium containing the starting composition to provide a medium comprising at least one target steviol glycoside.

The microorganism can be any microorganism possessing the necessary enzyme(s) for converting the starting composition to target steviol glycoside(s). These enzymes are encoded within the microorganism's genome.

Suitable microorganisms include, but are not limited to, E. coli, Saccharomyces sp., Aspergillus sp., Pichia sp., Bacillus sp., Yarrowia sp. etc.

In one embodiment, the microorganism is free when contacted with the starting composition.

In another embodiment, the microorganism is immobilized when contacted with the starting composition. For example, the microorganism may be immobilized to a solid support made from inorganic or organic materials. Non-limiting examples of solid supports suitable to immobilize the microorganism include derivatized cellulose or glass, ceramics, metal oxides or membranes. The microorganism may be immobilized to the solid support, for example, by covalent attachment, adsorption, cross-linking, entrapment or encapsulation.

In still another embodiment, the enzyme capable of converting the starting composition to the target steviol glycoside is secreted out of the microorganism and into the reaction medium.

The target steviol glycoside is optionally purified. Purification of the target steviol glycoside from the reaction medium can be achieved by at least one suitable method to provide a highly purified target steviol glycoside composition. Suitable methods include crystallization, separation by membranes, centrifugation, extraction (liquid or solid phase), chromatographic separation, HPLC (preparative or analytical) or a combination of such methods.

### Uses

Highly purified target glycoside(s), particularly steviolmonoside, steviolmonoside

A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention can be used “as-is” or in combination with other sweeteners, flavors, food ingredients and combinations thereof.

Non-limiting examples of flavors include, but are not limited to, lime, lemon, orange, fruit, banana, grape, pear, pineapple, mango, berry, bitter almond, cola, cinnamon, sugar, cotton candy, vanilla and combinations thereof.

Non-limiting examples of other food ingredients include, but are not limited to, acidulants, organic and amino acids, coloring agents, bulking agents, modified starches, gums, texturizers, preservatives, caffeine, antioxidants, emulsifiers, stabilizers, thickeners, gelling agents and combinations thereof.

Highly purified target glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention can be prepared in various polymorphic forms, including but not limited to hydrates, solvates, anhydrous, amorphous forms and combinations thereof.

Highly purified target glycoside(s) particularly, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be incorporated as a high intensity natural sweetener in foodstuffs, beverages, pharmaceutical compositions, cosmetics, chewing gums, table top products, cereals, dairy products, toothpastes and other oral cavity compositions, etc.

Highly purified target glycoside(s) particularly, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be employed as a sweetening compound as the sole sweetener, or it may be used together with at least one naturally occurring high intensity sweeteners such as rebaudioside A, rebaudioside A2, rebaudioside A3, rebaudioside B, rebaudioside C, rebaudioside C2, rebaudioside D, rebaudioside D2, rebaudioside F, rebaudioside F2, rebaudioside F3, rebaudioside G, rebaudioside H, rebaudioside I, rebaudioside 12, rebaudioside 13, rebaudioside J, rebaudioside K, rebaudioside K2, rebaudioside L, rebaudioside M, rebaudioside M2, rebaudioside N, rebaudioside O, rebaudioside O2, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside W, rebaudioside W2, rebaudioside W3, rebaudioside Y, rebaudioside Z1, rebaudioside Z2, dulcoside A, dulcoside C, stevioside D, stevioside E, stevioside E2, stevioside F, mogrosides, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, Luo Han Guo sweetener, mogroside V, siamenoside and combinations thereof.

In a particular embodiment, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be used in a sweetener composition comprising a compound selected from the group consisting of rebaudioside A, rebaudioside A2, rebaudioside A3, rebaudioside B, rebaudioside C, rebaudioside C2, rebaudioside D, rebaudioside D2, rebaudioside F, rebaudioside F2, rebaudioside F3, rebaudioside G, rebaudioside H, rebaudioside I, rebaudioside 12, rebaudioside 13, rebaudioside J, rebaudioside K, rebaudioside K2, rebaudioside L, rebaudioside M, rebaudioside M2, rebaudioside N, rebaudioside O, rebaudioside O2, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside W, rebaudioside W2, rebaudioside W3, rebaudioside Y, rebaudioside Z1, rebaudioside Z2, dulcoside A, dulcoside C, stevioside D, stevioside E, stevioside E2, stevioside F, NSF-02, Mogroside V, Luo Han Guo, allulose, allose, D-tagatose, erythritol and combinations thereof.

Highly purified target glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may also be used in combination with synthetic high intensity sweeteners such as sucralose, potassium acesulfame, aspartame, alitame, saccharin, neohesperidin dihydrochalcone, cyclamate, neotame, dulcin, suosan advantame, salts thereof, and combinations thereof.

Moreover, highly purified target steviol glycoside(s) particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be used in combination with natural sweetener suppressors such as gymnemic acid, hodulcin, ziziphin, lactisole, and others. Steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may also be combined with various umami taste enhancers. Steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be mixed with umami tasting and sweet amino acids such as glutamate, aspartic acid, glycine, alanine, threonine, proline, serine, glutamate, lysine, tryptophan and combinations thereof.

Highly purified target steviol glycoside(s) particularly, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be used in combination with one or more additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof.

Highly purified target steviol glycoside(s) particularly, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may be combined with polyols or sugar alcohols. The term “polyol” refers to a molecule that contains more than one hydroxyl group. A polyol may be a diol, triol, or a tetraol which contain 2, 3, and 4 hydroxyl groups, respectively. A polyol also may contain more than four hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group. Examples of polyols include, but are not limited to, erythritol, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols and sugar alcohols or any other carbohydrates capable of being reduced which do not adversely affect the taste of the sweetener composition.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may be combined with reduced calorie sweeteners such as, for example, D-tagatose, L-sugars, L-sorbose, L-arabinose and combinations thereof.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may also be combined with various carbohydrates. The term “carbohydrate” generally refers to aldehyde or ketone compounds substituted with multiple hydroxyl groups, of the general formula (CH₂O)ₙ, wherein n is 3-30, as well as their oligomers and polymers. The carbohydrates of the present invention can, in addition, be substituted or deoxygenated at one or more positions. Carbohydrates, as used herein, encompass unmodified carbohydrates, carbohydrate derivatives, substituted carbohydrates, and modified carbohydrates. As used herein, the phrases “carbohydrate derivatives”, “substituted carbohydrate”, and “modified carbohydrates” are synonymous. Modified carbohydrate means any carbohydrate wherein at least one atom has been added, removed, or substituted, or combinations thereof. Thus, carbohydrate derivatives or substituted carbohydrates include substituted and unsubstituted monosaccharides, disaccharides, oligosaccharides, and polysaccharides. The carbohydrate derivatives or substituted carbohydrates optionally can be deoxygenated at any corresponding C-position, and/or substituted with one or more moieties such as hydrogen, halogen, haloalkyl, carboxyl, acyl, acyloxy, amino, amido, carboxyl derivatives, alkylamino, dialkylamino, arylamino, alkoxy, aryloxy, nitro, cyano, sulfo, mercapto, imino, sulfonyl, sulfenyl, sulfinyl, sulfamoyl, carboalkoxy, carboxamido, phosphonyl, phosphinyl, phosphoryl, phosphino, thioester, thioether, oximino, hydrazino, carbamyl, phospho, phosphonato, or any other viable functional group provided the carbohydrate derivative or substituted carbohydrate functions to improve the sweet taste of the sweetener composition.

Examples of carbohydrates which may be used in accordance with this invention include, but are not limited to, psicose, turanose, allose, tagatose, trehalose, galactose, rhamnose, various cyclodextrins, cyclic oligosaccharides, various types of maltodextrins, dextran, sucrose, glucose, ribulose, fructose, threose, arabinose, xylose, lyxose, allose, altrose, mannose, idose, lactose, maltose, invert sugar, isotrehalose, neotrehalose, isomaltulose, erythrose, deoxyribose, gulose, idose, talose, erythrulose, xylulose, psicose, turanose, cellobiose, amylopectin, glucosamine, mannosamine, fucose, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), sorbose, nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, ribose, isomerized liquid sugars such as high fructose corn syrups, coupling sugars, and soybean oligosaccharides. Additionally, the carbohydrates as used herein may be in either the D- or L-configuration.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention can be used in combination with various physiologically active substances or functional ingredients. Functional ingredients generally are classified into categories such as carotenoids, dietary fiber, fatty acids, saponins, antioxidants, nutraceuticals, flavonoids, isothiocyanates, phenols, plant sterols and stanols (phytosterols and phytostanols); polyols; prebiotics, probiotics; phytoestrogens; soy protein; sulfides/thiols; amino acids;

proteins; vitamins; and minerals. Functional ingredients also may be classified based on their health benefits, such as cardiovascular, cholesterol-reducing, and anti-inflammatory. Exemplary functional ingredients are provided in WO2013/096420, the contents of which is hereby incorporated by reference.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be applied as a high intensity sweetener to produce zero calorie, reduced calorie or diabetic beverages and food products with improved taste characteristics. It may also be used in drinks, foodstuffs, pharmaceuticals, and other products in which sugar cannot be used. In addition, highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM can be used as a sweetener not only for drinks, foodstuffs, and other products dedicated for human consumption, but also in animal feed and fodder with improved characteristics.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be applied as a flavor modifier to produce zero calorie, reduced calorie or diabetic beverages and food products with modified flavor. When used as a flavor modifier, or a flavor with modifying properties (FMP), the highly purified target steviol glycoside is used in a consumable product below the detection level of the flavor modifier or FMP. The flavor modifier or FMP therefore does not impart a detectable taste or flavor of its own to the consumable product, but instead serves to modify the consumer's detection of tastes and/or flavors of other ingredients in the consumable product. One example of taste and flavor modification is sweetness enhancement, in which the flavor modifier or FMP itself does not contribute to the sweetness of the consumable product, but enhances the quality of the sweetness tasted by the consumer.

Examples of consumable products in which highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may be used as a flavor modifier or flavor with modifying properties include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

Highly purified target steviol glycoside(s), particularly steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained according to this invention may be applied as a foaming suppressor to produce zero calorie, reduced calorie or diabetic beverages and food products.

Examples of consumable products in which highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM may be used as a sweetening compound include, but are not limited to, alcoholic beverages such as vodka, wine, beer, liquor, and sake, etc.; natural juices; refreshing drinks; carbonated soft drinks; diet drinks; zero calorie drinks; reduced calorie drinks and foods; yogurt drinks; instant juices; instant coffee; powdered types of instant beverages; canned products; syrups; fermented soybean paste; soy sauce; vinegar; dressings; mayonnaise; ketchups; curry; soup; instant bouillon; powdered soy sauce; powdered vinegar; types of biscuits; rice biscuit; crackers; bread; chocolates; caramel; candy; chewing gum; jelly; pudding; preserved fruits and vegetables; fresh cream; jam; marmalade; flower paste; powdered milk; ice cream; sorbet; vegetables and fruits packed in bottles; canned and boiled beans; meat and foods boiled in sweetened sauce; agricultural vegetable food products; seafood; ham; sausage; fish ham; fish sausage; fish paste; deep fried fish products; dried seafood products; frozen food products; preserved seaweed; preserved meat; tobacco; medicinal products; and many others. In principle it can have unlimited applications.

During the manufacturing of products such as foodstuffs, drinks, pharmaceuticals, cosmetics, table top products, and chewing gum, the conventional methods such as mixing, kneading, dissolution, pickling, permeation, percolation, sprinkling, atomizing, infusing and other methods may be used.

Moreover, the highly purified target steviol glycoside(s) steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM obtained in this invention may be used in dry or liquid forms.

The highly purified target steviol glycoside can be added before or after heat treatment of food products. The amount of the highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM depends on the purpose of usage. As discussed above, it can be added alone or in combination with other compounds.

The present invention is also directed to sweetness enhancement in beverages using steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM. Accordingly, the present invention provides a beverage comprising a sweetener and steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM as a sweetness enhancer, wherein steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM is present in a concentration at or below their respective sweetness recognition thresholds.

As used herein, the term “sweetness enhancer” refers to a compound capable of enhancing or intensifying the perception of sweet taste in a composition, such as a beverage. The term “sweetness enhancer” is synonymous with the terms “sweet taste potentiator,” “sweetness potentiator,” “sweetness amplifier,” and “sweetness intensifier.”

The term “sweetness recognition threshold concentration,” as generally used herein, is the lowest known concentration of a sweet compound that is perceivable by the human sense of taste, typically around 1.0% sucrose equivalence (1.0% SE). Generally, the sweetness enhancers may enhance or potentiate the sweet taste of sweeteners without providing any noticeable sweet taste by themselves when present at or below the sweetness recognition threshold concentration of a given sweetness enhancer; however, the sweetness enhancers may themselves provide sweet taste at concentrations above their sweetness recognition threshold concentration. The sweetness recognition threshold concentration is specific for a particular enhancer and can vary based on the beverage matrix. The sweetness recognition threshold concentration can be easily determined by taste testing increasing concentrations of a given enhancer until greater than 1.0% sucrose equivalence in a given beverage matrix is detected. The concentration that provides about 1.0% sucrose equivalence is considered the sweetness recognition threshold.

In some embodiments, sweetener is present in the beverage in an amount from about 0.5% to about 12% by weight, such as, for example, about 1.0% by weight, about 1.5% by weight, about 2.0% by weight, about 2.5% by weight, about 3.0% by weight, about 3.5% by weight, about 4.0% by weight, about 4.5% by weight, about 5.0% by weight, about 5.5% by weight, about 6.0% by weight, about 6.5% by weight, about 7.0% by weight, about 7.5% by weight, about 8.0% by weight, about 8.5% by weight, about 9.0% by weight, about 9.5% by weight, about 10.0% by weight, about 10.5% by weight, about 11.0% by weight, about 11.5% by weight or about 12.0% by weight.

In a particular embodiment, the sweetener is present in the beverage in an amount from about 0.5% of about 10%, such as for example, from about 2% to about 8%, from about 3% to about 7% or from about 4% to about 6% by weight. In a particular embodiment, the sweetener is present in the beverage in an amount from about 0.5% to about 8% by weight. In another particular embodiment, the sweetener is present in the beverage in an amount from about 2% to about 8% by weight.

In one embodiment, the sweetener is a traditional caloric sweetener. Suitable sweeteners include, but are not limited to, sucrose, fructose, glucose, high fructose corn syrup and high fructose starch syrup.

In another embodiment, the sweetener is erythritol.

In still another embodiment, the sweetener is a rare sugar. Suitable rare sugars include, but are not limited to, D-allose, D-psicose, D-ribose, D-tagatose, L-glucose, L-fucose, L-arabinose, D-turanose, D-leucrose and combinations thereof.

It is contemplated that a sweetener can be used alone, or in combination with other sweeteners.

In one embodiment, the rare sugar is D-allose. In a more particular embodiment, D-allose is present in the beverage in an amount of about 0.5% to about 10% by weight, such as, for example, from about 2% to about 8%.

In another embodiment, the rare sugar is D-psicose. In a more particular embodiment, D-psicose is present in the beverage in an amount of about 0.5% to about 10% by weight, such as, for example, from about 2% to about 8%.

In still another embodiment, the rare sugar is D-ribose. In a more particular embodiment, D-ribose is present in the beverage in an amount of about 0.5% to about 10% by weight, such as, for example, from about 2% to about 8%.

In yet another embodiment, the rare sugar is D-tagatose. In a more particular embodiment, D-tagatose is present in the beverage in an amount of about 0.5% to about 10% by weight, such as, for example, from about 2% to about 8%.

In a further embodiment, the rare sugar is L-glucose. In a more particular embodiment, L-glucose is present in the beverage in an amount of about 0.5% to about 10% by weight, such as, for example, from about 2% to about 8%.

In one embodiment, the rare sugar is L-fucose. In a more particular embodiment, L-fucose is present in the beverage in an amount of about 0.5% to about 10% by weight, such as, for example, from about 2% to about 8%.

In another embodiment, the rare sugar is L-arabinose. In a more particular embodiment, L-arabinose is present in the beverage in an amount of about 0.5% to about 10% by weight, such as, for example, from about 2% to about 8%.

In yet another embodiment, the rare sugar is D-turanose. In a more particular embodiment, D-turanose is present in the beverage in an amount of about 0.5% to about 10% by weight, such as, for example, from about 2% to about 8%.

In yet another embodiment, the rare sugar is D-leucrose. In a more particular embodiment, D-leucrose is present in the beverage in an amount of about 0.5% to about 10% by weight, such as, for example, from about 2% to about 8%.

The addition of the sweetness enhancer at a concentration at or below its sweetness recognition threshold increases the detected sucrose equivalence of the beverage comprising the sweetener and the sweetness enhancer compared to a corresponding beverage in the absence of the sweetness enhancer. Moreover, sweetness can be increased by an amount more than the detectable sweetness of a solution containing the same concentration of the at least one sweetness enhancer in the absence of any sweetener.

Accordingly, the present invention also provides a method for enhancing the sweetness of a beverage comprising a sweetener comprising providing a beverage comprising a sweetener and adding a sweetness enhancer selected from steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM or a combination thereof, wherein steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM are present in a concentration at or below their sweetness recognition thresholds.

Addition of steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM in a concentration at or below the sweetness recognition threshold to a beverage containing a sweetener may increase the detected sucrose equivalence from about 1.0% to about 5.0%, such as, for example, about 1.0%, about 1.5%, about 2.0%, about 2.5%, about 3.0%, about 3.5%, about 4.0%, about 4.5% or about 5.0%.

The following examples illustrate preferred embodiments of the invention for the preparation of highly purified target steviol glycoside(s), particularly steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3 and/or rebaudioside AM. It will be understood that the invention is not limited to the materials, proportions, conditions and procedures set forth in the examples, which are only illustrative.

### EXAMPLES

### Example 1

### Protein Sequences of Engineered Enzymes Used in the Biocatalytic Process

SEQ ID 1:
>SuSy_At, variant PM1-54-2-E05 (engineered sucrose
synthase; source of WT gene: Arabidopsis thaliana)
MANAERMITRVHSQRERLNETLVSERNEVLALLSRVEAKGKGILQQNQII
AEFEALPEQTRKKLEGGPFFDLLKSTQEAIVLPPWVALAVRPRPGVWEYL
RVNLHALVVEELQPAEFLHFKEELVDGVKNGNFTLELDFEPFNASIPRPT
LHKYIGNGVDFLNRHLSAKLFHDKESLLPLLDFLRLHSHQGKNLMLSEKI
QNLNTLQHTLRKAEEYLAELKSETLYEEFEAKFEEIGLERGWGDNAERVL
DMIRLLLDLLEAPDPSTLETFLGRVPMVFNVVILSPHGYFAQDNVLGYPD
TGGQVVYILDQVRALEIEMLQRIKQQGLNIKPRILILTRLLPDAVGTTCG
ERLERVYDSEYCDILRVPFRTEKGIVRKWISRFEVWPYLETYTEDAAVEL
SKELNGKPDLIIGNYSDGNLVASLLAHKLGVTQCTIAHALEKTKYPDSDI
YWKKLDDKYHFSCQFTADIFAMNHTDFIITSTFQEIAGSKETVGQYESHT
AFTLPGLYRVVHGIDVFDPKFNIVSPGADMSIYFPYTEEKRRLTKFHSEI
EELLYSDVENDEHLCVLKDKKKPILFTMARLDRVKNLSGLVEWYGKNTRL
RELVNLVVVGGDRRKESKDNEEKAEMKKMYDLIEEYKLNGQFRWISSQMD
RVRNGELYRYICDTKGAFVQPALYEAFGLTVVEAMTCGLPTFATCKGGPA
EIIVHGKSGFHIDPYHGDQAADLLADFFTKCKEDPSHWDEISKGGLQRIE
EKYTWQIYSQRLLTLTGVYGFWKHVSNLDRLEHRRYLEMFYALKYRPLAQ
AVPLAQDD
SEQ ID 2:
>UGTS12 variant 0234 (engineered glucosyltrans-
ferase; source of WT gene: Solanum lycopersicum)
MATNLRVLMFPWLAYGHISPFLNIAKQLADRGFLIYLCSTRINLESIIKK
IPEKYADSIHLIELQLPELPELPPHYHTTNGLPPHLNPTLHKALKMSKPN
FSRILQNLKPDLLIYDVLQPWAEHVANEQGIPAGKLLVSCAAVFSYFFSF
RKNPGVEFPFPAIHLPEVEKVKIREILAKEPEEGGRLDEGNKQMMLMCTS
RTIEAKYIDYCTELCNWKVVPVGPPFQDLITNDADNKELIDWLGTKPENS
TVFVSFGSEYFLSKEDMEEIAFALEASNVNFIWVVRFPKGEERNLEDALP
EGFLERIGERGRVLDKFAPQPRILNHPSTGGFISHCGWNSVMESIDFGVP
IIAMPIHNDQPINAKLMVELGVAVEIVRDDDGKIHRGEIAEALKSVVTGE
TGEILRAKVREISKNLKSIRDEEMDAVAEELIQLCRNSNKSK
SEQ ID 3:
>UGT76G1 variant 0042 (engineered glucosyltrans-
ferase; source of WT gene: Stevia rebaudiana)
MENKTETTVRRRRRIILFPVPFQGHINPILQLANVLYSKGFAITILHTNF
NKPKTSNYPHFTFRFILDNDPQDERISNLPTHGPLAGMRIPIINEHGADE
LRRELELLMLASEEDEEVSCLITDALWYFAQDVADSLNLRRLVLMTSSLF
NFHAHVSLPQFDELGYLDPDDKTRLEEQASGFPMLKVKDIKSAYSNWQIG
KEILGKMIKQTKASSGVIWNSFKELEESELETVIREIPAPSFLIPLPKHL
TASSSSLLDHDRTVFEWLDQQAPSSVLYVSFGSTSEVDEKDFLEIARGLV
DSGQSFLWVVRPGFVKGSTWVEPLPDGFLGERGKIVKWVPQQEVLAHPAI
GAFWTHSGWNSTLESVCEGVPMIFSSFGGDQPLNARYMSDVLRVGVYLEN
GWERGEVVNAIRRVMVDEEGEYIRQNARVLKQKADVSLMKGGSSYESLES
LVSYISSL

### Example 2

### Expression and Formulation of SuSy_At Variant of SEQ ID 1

The gene coding for the SuSy_At variant of SEQ ID 1 (EXAMPLE 1) was cloned into the expression vector pLE1A17 (derivative of pRSF-1b, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/l) at 37° C. Expression of the genes was induced at logarithmic phase by IPTG (0.2 mM) and carried out at 30° C. and 200 rpm for 16-18 hours. Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with distilled water, resulting in an enzymatic active preparation.

For enzymatic active preparations of SuSy_At, activity in Units is defined as follows: 1 mU of SuSy_At turns over 1 nmol of sucrose into fructose in 1 minute. Reaction conditions for the assay are 30° C., 50 mM potassium phosphate buffer pH 7.0, 400 mM sucrose at to, 3 mM MgCl₂, and 15 mM uridine diphosphate (UDP).

### Example 3

### Expression and Formulation of UGTS12 Variant of SEQ ID 2

The gene coding for the UGTS12 variant of SEQ ID 2 (EXAMPLE 1) was cloned into the expression vector pLE1A17 (derivative of pRSF-lb, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/l) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGTS12, activity in Units is defined as follows: 1 mU of UGTS12 turns over 1 nmol of rebaudioside A (RebA) into rebaudioside D (Reb D) in 1 minute. Reaction conditions for the assay are 30° C., 50 mM potassium phosphate buffer pH 7.0, 10 mM RebA at to, 500 mM sucrose, 3 mM MgCl₂, 0.25 mM uridine diphosphate (UDP) and 3 U/mL of SuSy_At.

### Example 4

### Expression and Formulation of UGT76G1 Variant of SEQ ID 3

The gene coding for the UGT76G1 variant of SEQ ID 3 (EXAMPLE 1) was cloned into the expression vector pLE1A17 (derivative of pRSF-lb, Novagen). The resulting plasmid was used for transformation of E. coli BL21(DE3) cells.

Cells were cultivated in ZYM505 medium (F. William Studier, Protein Expression and Purification 41 (2005) 207-234) supplemented with kanamycin (50 mg/l) at 37° C. Expression of the genes was induced at logarithmic phase by IPTG (0.1 mM) and carried out at 30° C. and 200 rpm for 16-18 hours.

Cells were harvested by centrifugation (3220×g, 20 min, 4° C.) and re-suspended to an optical density of 200 (measured at 600 nm (OD₆₀₀)) with cell lysis buffer (100 mM Tris-HCl pH 7.0; 2 mM MgCl₂, DNA nuclease 20 U/mL, lysozyme 0.5 mg/mL). Cells were then disrupted by sonication and crude extracts were separated from cell debris by centrifugation (18000×g 40 min, 4° C.). The supernatant was sterilized by filtration through a 0.2 μm filter and diluted 50:50 with 1 M sucrose solution, resulting in an enzymatic active preparation.

For enzymatic active preparations of UGT76G1, activity in Units is defined as follows: 1 mU of UGT76G1 turns over 1 nmol of rebaudioside D (Reb D) into rebaudioside M (Reb M) in 1 minute. Reaction conditions for the assay are 30° C., 50 mM potassium phosphate buffer pH 7.0, 10 mM RebA at to, 500 mM sucrose, 3 mM MgCl₂, 0.25 mM uridine diphosphate (UDP) and 3 U/mL of SuSy_At.

### Example 5

Synthesis of Rebaudioside AM from Stevioside in a One-Pot Reaction, Adding UGTS12, SuSy_At and UGT76G1 at the Same Time

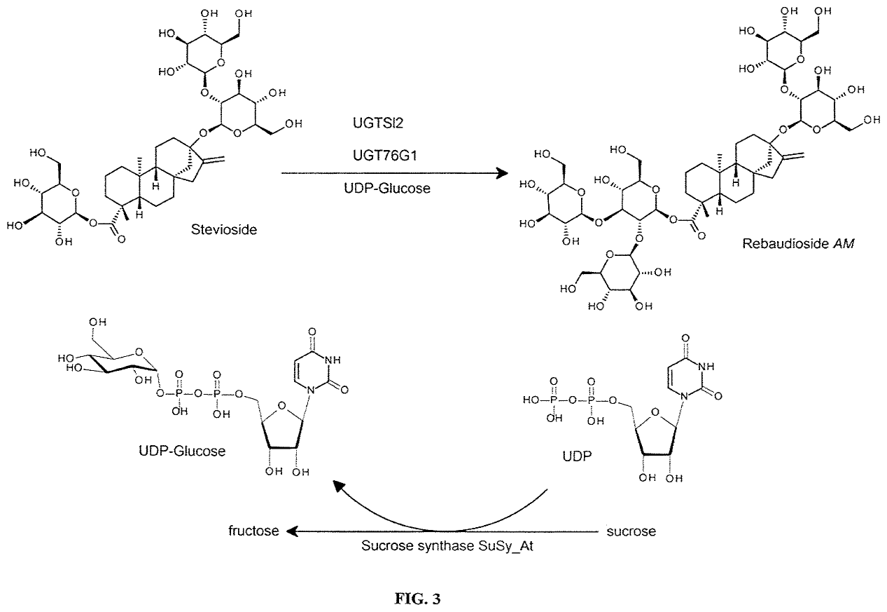

Rebaudioside AM (reb AM) was synthesized directly from stevioside in a one-pot reaction (FIG. 3), utilizing the three enzymes (see EXAMPLES 1, 2, 3 and 4): UGTS12 (variant of SEQ ID 2), SuSy_At-(variant of SEQ ID 1) and UGT76G1 (variant of SEQ ID 3). The final reaction solution contained 105 U/L UGTS12, 405 U/L SuSy_At, 3 U/L UGT76G1, 5 mM stevioside, 0.25 mM uridine diphosphate (UDP), 1 M sucrose, 4 mM MgCl₂ and potassium phosphate buffer (pH 6.6). First, 207 mL of distilled water were mixed with 0.24 g MgCl₂.6H20, 103 g sucrose, 9.9 mL of 1.5 M potassium phosphate buffer (pH 6.6) and 15 g stevioside. After dissolving the components, the temperature was adjusted to 45° C. and UGTS12, SuSy_At, UGT76G1 and 39 mg UDP were added. The reaction mixture was incubated at 45° C. shaker for 24 hrs. Additional 39 mg UDP was added at 8 hrs and 18 hours. The content of reb AM, reb E, stevioside, reb M, reb B, steviolbioside and reb I at several time points was analyzed by HPLC.

For analysis, biotransformation samples were inactivated by adjusting the reaction mixture to pH5.5 using 17% H₃PO₄ and then boiled for 10 minutes. Resulting samples were filtered, the filtrates were diluted 10 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB-C18, 4.6 mm×150 mm, 2.7 μm at 40° C. The mobile phase consisted of two premixes:
premix 1 containing 75% 10 mM phosphate buffer (pH2.6) and 25% acetonitrile, and
premix 2 containing 68% 10 mM phosphate buffer (pH2.6) and 32% acetonitrile.

Elution gradient started with premix 1, changed to premix 2 to 50% at 12.5 minute, changed to premix 2 to 100% at 13 minutes. Total run time was 45 minutes. The column temperature was maintained at 40° C. The injection volume was 5 μL. Rebaudioside species were detected by UV at 210 nm.

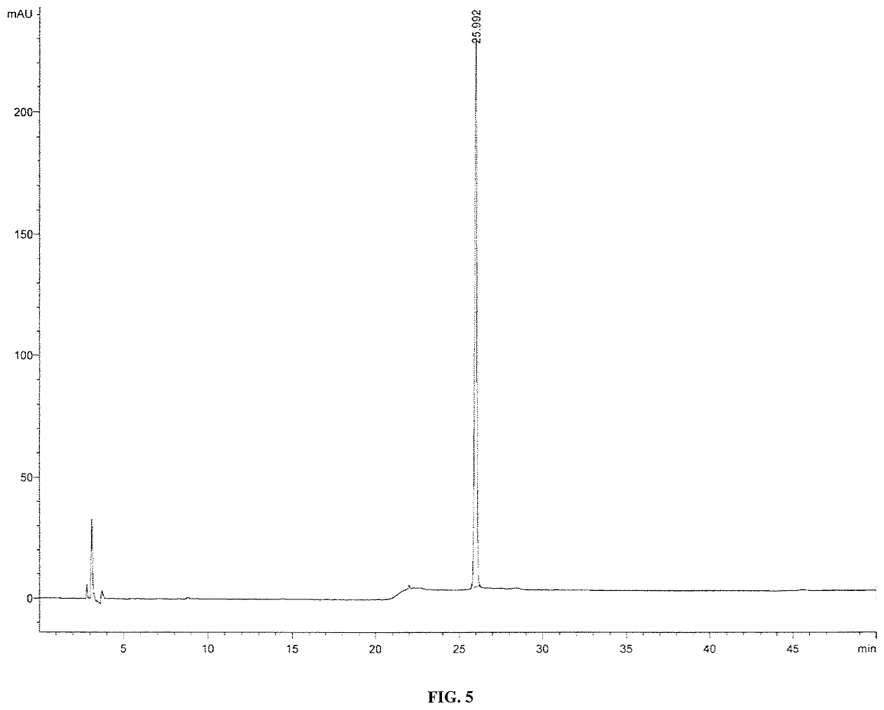

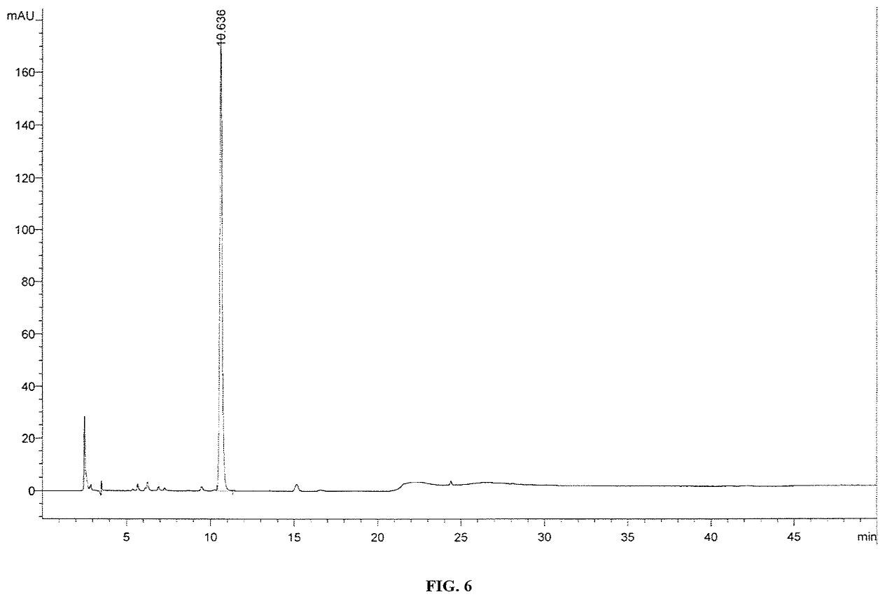

Table 3 shows for each time point the conversion of stevioside into identified rebaudioside species (area percentage). The chromatograms of stevioside and the reaction mixture at 24 hours are shown in FIG. 5 and FIG. 6, respectively. Those with skill in the art will appreciate that retention times can occasionally vary with changes in solvent and/or equipment.

TABLE 3
Biotransformation of stevioside to reb AM

| Time, | % conversion from stevioside | | | | | | |
|---|---|---|---|---|---|---|---|
| hrs | Reb E | Reb AM | Reb M | Reb I | Stevioside | Reb B | Steviolbioside |
| 0 | 0 | 0 | 0 | 0 | 100 | 0 | 0 |
| 6 | 1.9 | 35.9 | 1.3 | 1.7 | 58.7 | 0.0 | 0.4 |
| 18 | 0.9 | 96.7 | 1.3 | 0.6 | 0.0 | 0.0 | 0.4 |
| 24 | 0.3 | 96.4 | 2.1 | 0.7 | 0.0 | 0.2 | 0.4 |

### Example 6

Synthesis of Rebaudioside AM from Rebaudioside E in a One-Pot Reaction, SuSy_At and UGT76G1 at the Same Time

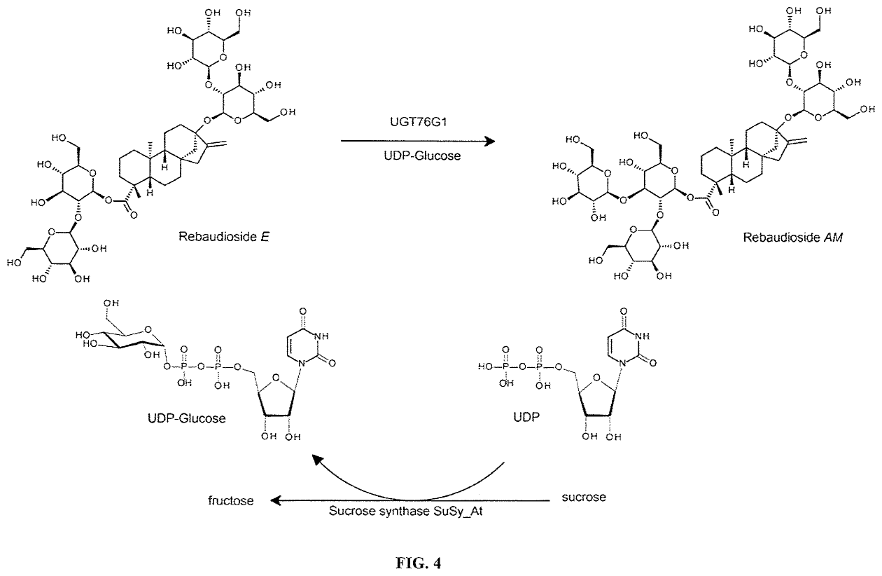

Rebaudioside AM (reb AM) was synthesized directly from rebaudioside E (reb E) in a one-pot reaction (FIG. 4), utilizing the two enzymes (see EXAMPLES 1, 2 and 4): SuSy_At-(variant of SEQ ID 1) and UGT76G1 (variant of SEQ ID 3). The final reaction solution contained 405 U/L SuSy_At, 3 U/L UGT76G1, 5 mM reb E, 0.25 mM uridine diphosphate (UDP), 1 M sucrose, 4 mM MgCl₂.6H₂O and potassium phosphate buffer (pH 6.6). First, 37 mL of distilled water were mixed with 40.3 mg MgCl₂, 17.12 g sucrose, 1.65 mL of 1.5 M potassium phosphate buffer (pH 6.6) and 5.04 g reb E. After dissolving the components, the temperature was adjusted to 45° C. and SuSy_At, UGT76G1 and 6.5 mg UDP were added. The reaction mixture was incubated at 45° C. shaker for 24 hrs. Additional 6.5 mg UDP was added at 8 hrs and 18 hours. The content of reb AM, reb E, stevioside, reb A, reb M, reb B, and steviolbioside at several time points was analyzed by HPLC.

For analysis, biotransformation samples were inactivated by adjusting the reaction mixture to pH5.5 using 17% H₃PO₄ and then boiled for 10 minutes. Resulting samples were filtered, the filtrates were diluted 10 times and used as samples for HPLC analysis. HPLC assay was carried out on Agilent HP 1200 HPLC system, comprised of a pump, a column thermostat, an auto sampler, a UV detector capable of background correction and a data acquisition system. Analytes were separated using Agilent Poroshell 120 SB-C18, 4.6 mm×150 mm, 2.7 μm at 40° C. The mobile phase consisted of two premixes:
premix 1 containing 75% 10 mM phosphate buffer (pH2.6) and 25% acetonitrile, and
premix 2 containing 68% 10 mM phosphate buffer (pH2.6) and 32% acetonitrile.

Elution gradient started with premix 1, changed to premix 2 to 50% at 12.5 minute, changed to premix 2 to 100% at 13 minutes. Total run time was 45 minutes. The column temperature was maintained at 40° C. The injection volume was 5 μL. Rebaudioside species were detected by UV at 210 nm.

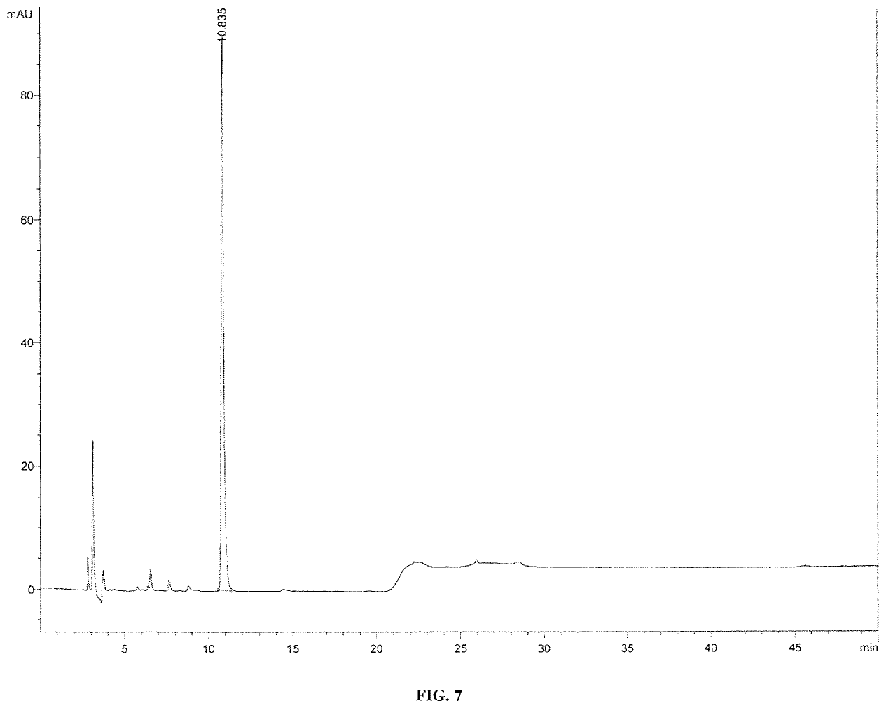

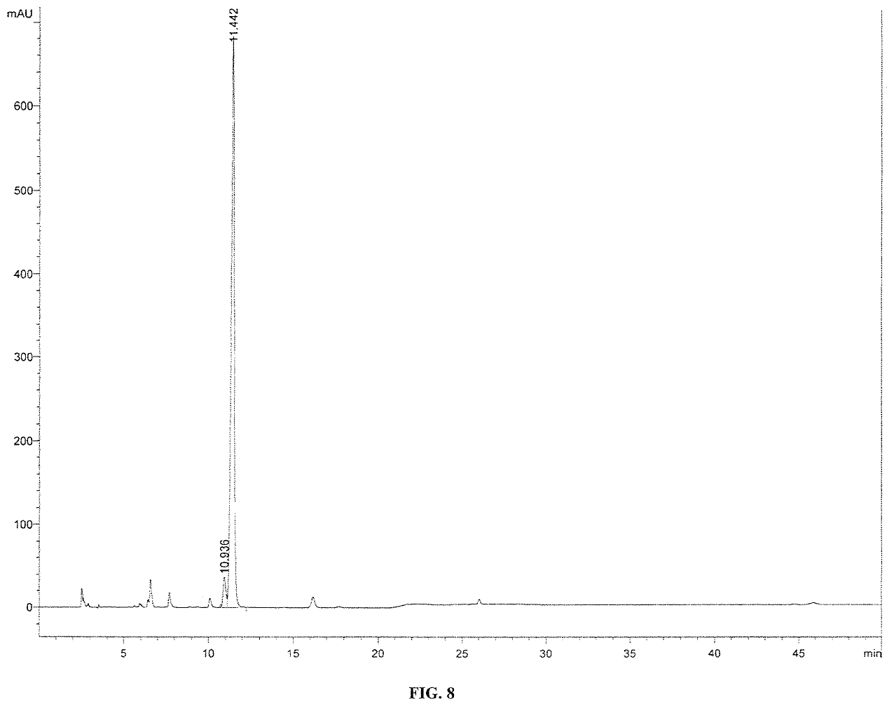

Table 4 shows for each time point the conversion of reb E into identified rebaudioside species (area percentage). The chromatograms of reb E and the reaction mixture at 24 hours are shown in FIG. 7 and FIG. 8, respectively. Those with skill in the art will appreciate that retention times can occasionally vary with changes in solvent and/or equipment.

TABLE 4
Biotransformation of reb E to reb AM

| | % conversion from Reb E | | | | | | |
|---|---|---|---|---|---|---|---|
| Time, | | | | | | | Steviol- |
| hrs | Reb E | Reb AM | Reb M | Reb A | Stevioside | Reb B | bioside |
| 0 | 99.46 | 0 | 0 | 0.54 | 0 | 0 | 0 |
| 4 | 40.75 | 57.92 | 0 | 0.59 | 0 | 0.73 | 0 |
| 7 | 24.79 | 73.92 | 0 | 0.58 | 0 | 0.71 | 0 |
| 24 | 4.38 | 94.33 | 0 | 0.59 | 0 | 0.70 | 0 |

### Example 7

### Purification of Rebaudioside AM

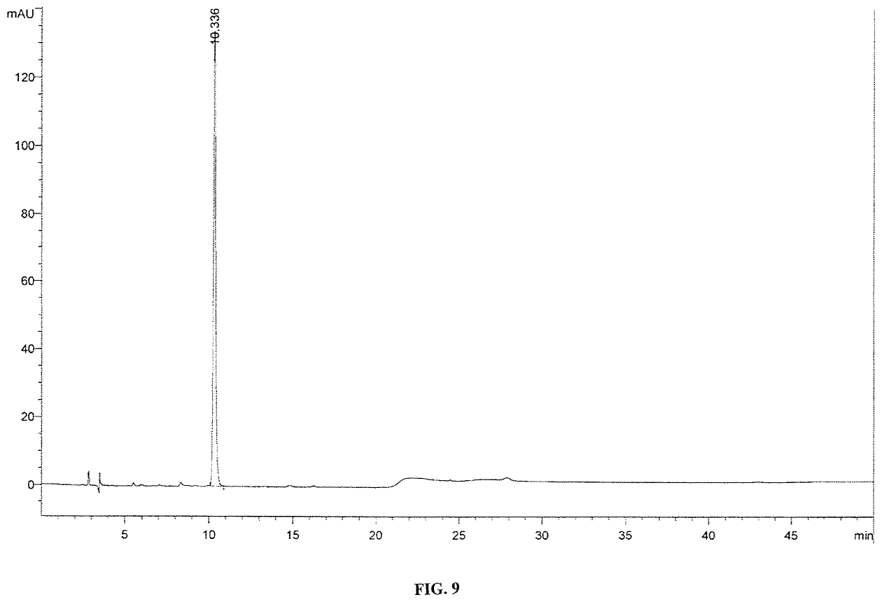

The reaction mixture of EXAMPLE 5, after 24 hrs, was inactivated by adjusting the pH to pH 5.5 with H₃PO₄ and then boiled for 10 minutes. After boiling the reaction mixture was filtered and diluted with RO water to 5% solids content. The diluted solution was passed through 1 L column packed with YWD03 macroporous adsorption resin (Cangzhou Yuanwei, China). Adsorbed steviol glycosides were eluted with 5 L 70% ethanol. The obtained eluate was evaporated until dryness to yield 16 g of dry powder which was dissolved in 80 mL of 70% methanol. The solution was crystallized at 20° C. for 3 days. The crystals were separated by filtration and dried in vacuum oven at 80° C. for 18 hours to yield 10.4 g of pure reb AM crystals with 95.92% purity, determined by HPLC assay. The chromatogram of reb AM is shown in FIG. 9. Those with skill in the art will appreciate that retention times can occasionally vary with changes in solvent and/or equipment.

### Example 8

### Structure Elucidation of Rebaudioside AM

NMR experiments were performed on a Bruker 500 MHz spectrometer, with the sample dissolved in pyridine-d5. Along with signals from the sample, signals from pyridine-d5 at δ_{C} 123.5, 135.5, 149.9 ppm and δ_{H} 7.19, 7.55, 8.71 ppm were observed.

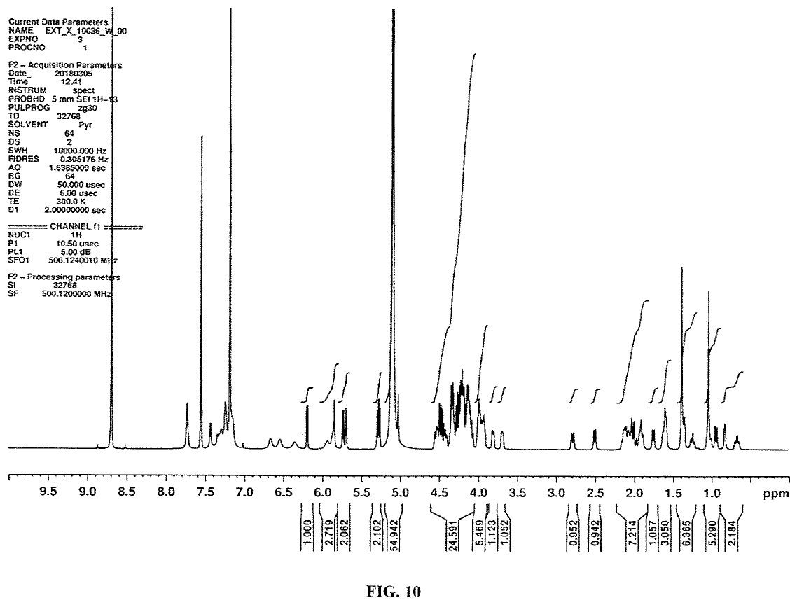

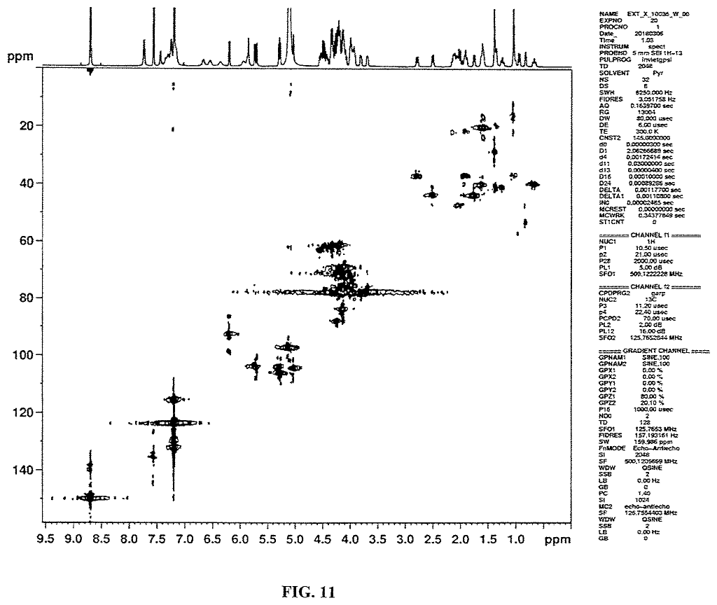

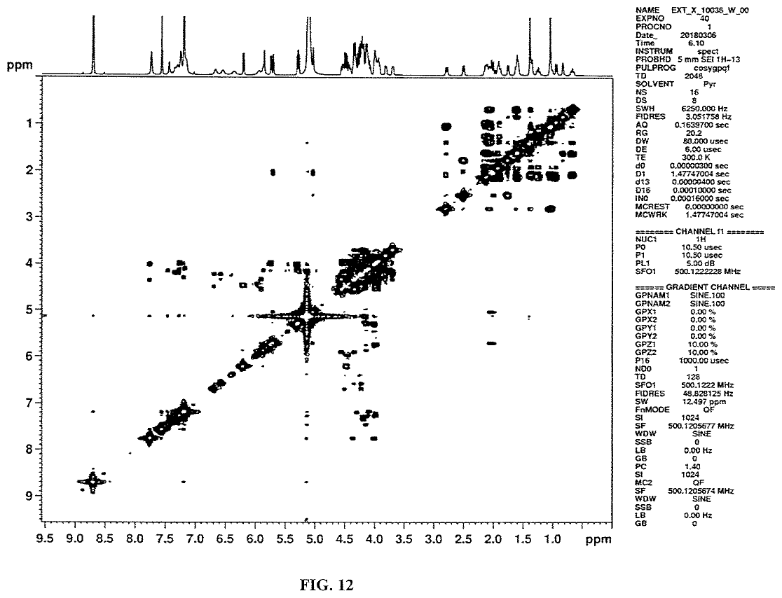

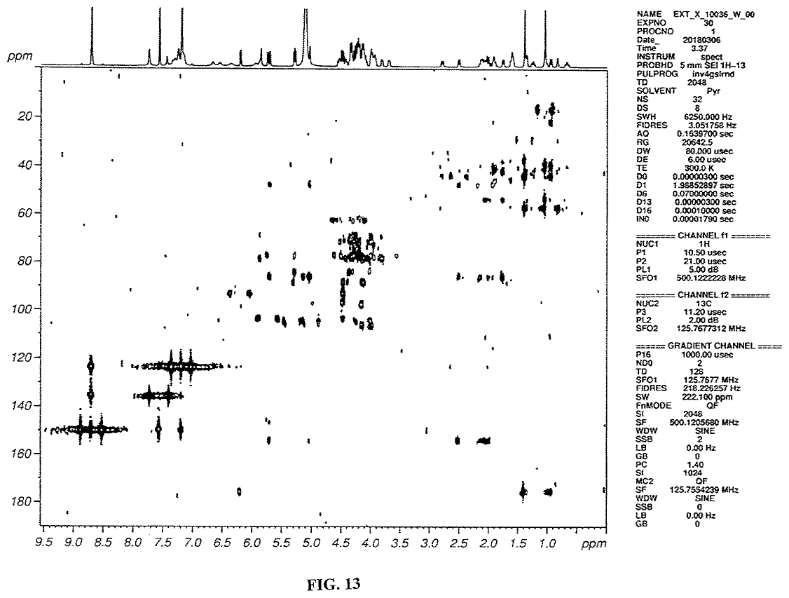

¹H-NMR-spectrum of rebaudioside AM in pyridine-d5 reveal the excellent quality of the sample (see FIG. 10). The HSQC (see FIG. 11) shows the presence of an exo-methylene group in the sugar region with a long-range coupling to C-15, observable in the H,H-COSY (FIG. 12). Other deep-fielded signals of the quaternary carbons (C-13, C-16 and C-19) are detected by the HMBC (FIG. 13). Correlation of the signals in the HSQC, HMBC and H,H-COSY reveal the presence of steviol glycoside with the following aglycone structure:

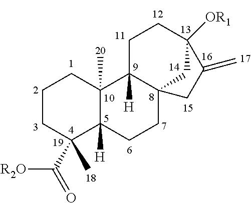

Correlation of HSQC and HMBC signals reveal five anomeric signals. The coupling constant of the anomeric protons of about 8 Hz and the broad signals of their sugar linkage allows the identification of these five sugars as β-D-glucopyranosides.

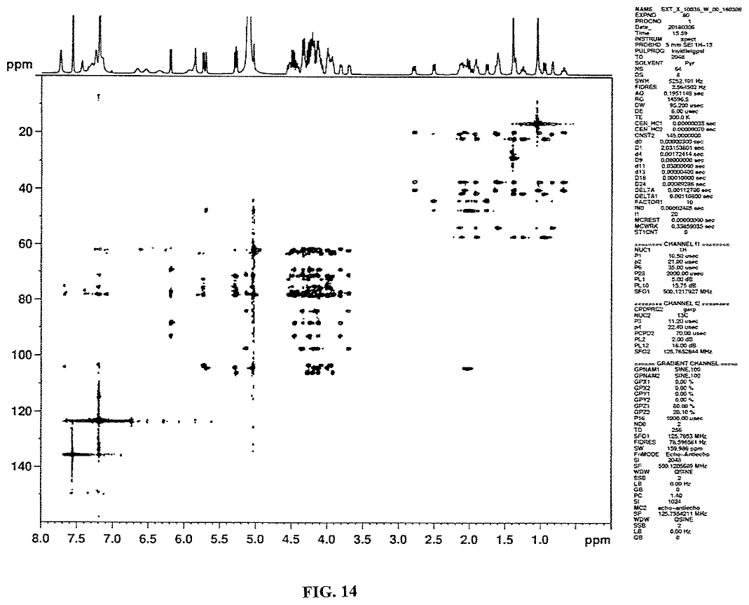

The observation of the anomeric protons in combination with HSQC and HMBC reveal the sugar linkage and the correlation to the aglycone. The assignment of the sugar sequence was confirmed by using the combination of HSQC-TOCSY (FIG. 14) and HSQC.

The NMR experiments above were applied to assign the chemical shifts of the protons and carbons, main coupling constants and main HMBC correlations (see Table 5).

TABLE 5
Chemical shifts of rebaudioside AM

| | | | | |
|---|---|---|---|---|
| Position | δ_{C} [ppm] | δ_{H} [ppm] | J [Hz] | HMBC (H → C) |

Aglycone moiety

| 1 | 39.9 | t | 0.68 | m | | |
|---|---|---|---|---|---|---|
| | | | 1.64 | m | | |
| 2 | 19.4 | t | 1.39 | m | | |
| | | | 2.08 | m | | |
| 3 | 37.4 | t | 1.05 | m | | |
| | | | 2.80 | m | | |
| 4 | 44.2 | s | — | | | |
| 5 | 57.3 | d | 0.95 | m | | |
| 6 | 21.7 | t | 1.90 | m | | |
| | | | 2.12 | m | | |
| 7 | 41.0 | t | 1.26 | m | | |
| | | | 1.38 | m | | |
| 8 | 41.9 | s | — | | | |
| 9 | 53.3 | d | 0.85 | m | | |
| 10 | 39.2 | s | — | | | |
| 11 | 20.1 | t | 1.59 | m | | |
| | | | 1.61 | m | | |
| 12 | 36.9 | t | 1.65 | m | | |
| | | | 1.92 | m | | |
| 13 | 85.9 | s | — | | | |
| 14 | 43.8 | t | 1.78 | d | 11.0 | |
| | | | 2.52 | d | 11.0 | |
| 15 | 47.4 | t | 2.00 | d | 16.0 | 7, 8, 9, 14 |
| | | | 2.06 | d | 16.0 | |
| 16 | 154.6 | s | — | | | |
| 17 | 104.3 | t | 5.03 | br s | | 13, 15, 16 |
| | | | 5.71 | br s | | |
| 18 | 28.5 | q | 1.40 | s | | 3, 4, 5, 19 |
| 19 | 175.2 | s | — | | | |
| 20 | 16.2 | q | 1.06 | s | | 1, 5, 9, 10 |

Sugar moiety
Sugar I: β-D-Glucopyranoside

| | | | | | | |
|---|---|---|---|---|---|---|
| 1ⁱ | 97.5 | d | 5.13 | d | 7.7 | 13 |
| 2ⁱ | 84.0 | d | 4.14 | m | | |
| 3ⁱ | 77.6 | d | 4.20 | m | | |
| 4ⁱ | 71.3 | d | 4.19 | m | | |
| 5ⁱ | 77.6 | d | 3.70 | m | | |
| 6ⁱ | 62.0 | t | 4.23 | m | | |
| | | | 4.32 | m | | |

Sugar II: β-D-Glucopyranoside

| | | | | | | |
|---|---|---|---|---|---|---|
| 1ⁱⁱ | 106.3 | d | 5.26 | d | 8.0 | 2* |
| 2ⁱⁱ | 76.8 | d | 4.13 | m | | |
| 3ⁱⁱ | 77.3 | d | 4.21 | m | | |
| 4ⁱⁱ | 71.6 | d | 4.18 | m | | |
| 5ⁱⁱ | 77.9 | d | 3.91 | m | | |
| 6ⁱⁱ | 62.4 | t | 4.29 | m | | |
| | | | 4.41 | m | | |

Sugar III: β-D-Glucopyranoside

| | | | | | | |
|---|---|---|---|---|---|---|
| 1ⁱⁱⁱ | 92.9 | d | 6.20 | d | 8.1 | 19 |
| 2ⁱⁱⁱ | 77.0 | d | 4.46 | m | | |
| 3ⁱⁱⁱ | 88.1 | d | 4.24 | m | | |
| 4ⁱⁱⁱ | 69.0 | d | 4.12 | m | | |
| 5ⁱⁱⁱ | 78.4 | d | 3.82 | m | | |
| 6ⁱⁱⁱ | 61.3 | t | 4.20 | m | | |
| | | | 4.33 | m | | |

Sugar IV: β-D-Glucopyranoside

| | | | | | | |
|---|---|---|---|---|---|---|
| 1^{iv} | 103.4 | d | 5.73 | d | 7.7 | 2ⁱⁱⁱ |
| 2^{iv} | 75.4 | d | 3.98 | m | | |
| 3^{iv} | 78.1 | d | 4.09 | m | | |
| 4^{iv} | 72.6 | d | 4.08 | m | | |
| 5^{iv} | 77.4 | d | 3.92 | m | | |
| 6^{iv} | 62.9 | t | 4.32 | m | | |
| | | | 4.51 | m | | |

Sugar V: β-D-Glucopyranoside

| | | | | | | |
|---|---|---|---|---|---|---|
| 1^{v} | 104.4 | d | 5.29 | d | 8.1 | 3ⁱⁱⁱ |
| 2^{v} | 75.1 | d | 4.00 | m | | |
| 3^{v} | 78.2 | d | 4.24 | m | | |
| 4^{v} | 71.4 | d | 4.27 | m | | |
| 5^{v} | 78.2 | d | 3.99 | m | | |
| 6^{v} | 61.9 | t | 4.27 | m | | |
| | | | 4.48 | m | | |

Correlation of all NMR data indicates rebaudioside having five β-D-glucopyranoses attached to a steviol aglycone, as depicted with the following chemical structure:

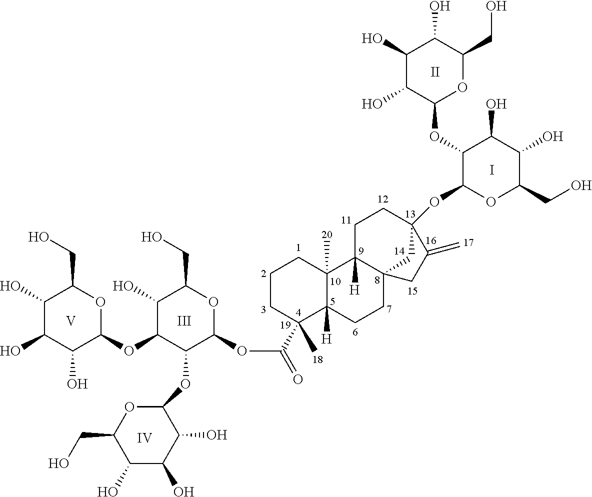

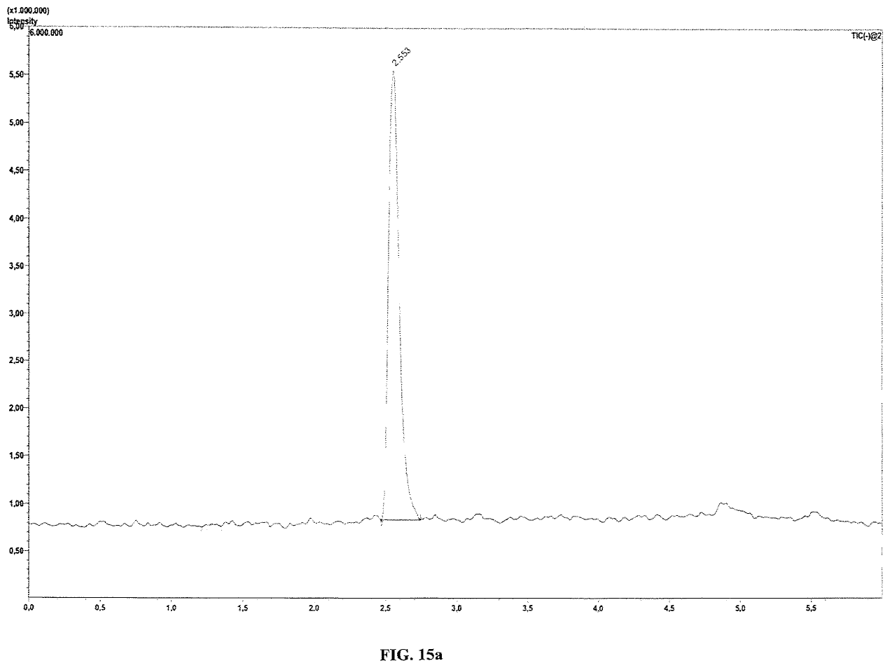

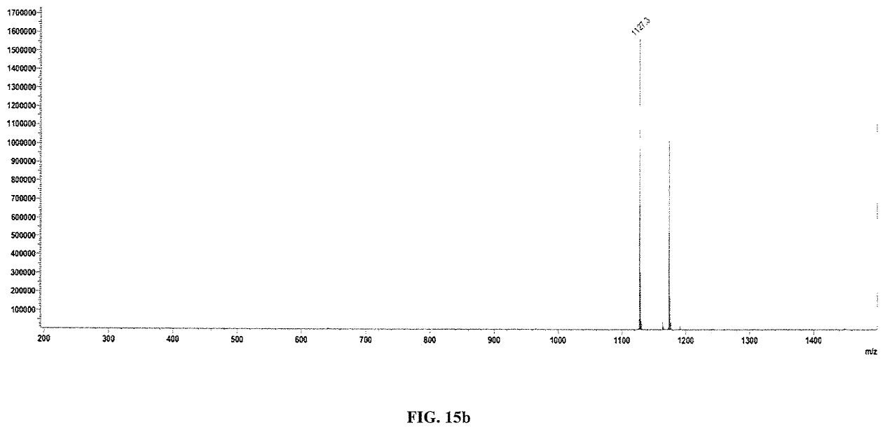

The chemical formula of rebaudioside AM is C54180028, which corresponds to a calculated monoisotopic molecular mass of 1128.5. For LCMS analysis, rebaudioside AM was dissolved in methanol and analyzed using Shimadzu Nexera 2020 UFLC LCMS instrument on a Cortecs UPLC C18 1.6 μm, 50×2.1 mm column. The observed LCMS (negative ESI mode) result of 1127.3 (see FIG. 15a and FIG. 15b respectively) is consistent with rebaudioside AM and corresponds to the ion (M−H)⁻.

### Solubility, Sweetness and Flavor Modification Properties of Reb AM

### Example 9

Reb AM was evaluated for it solubility and solution stability properties. Tables 6a and 6b, below, show the composition of the test sample, with the total steviol glycoside (TSG) percentage shown in the final column of Table 6b.

TABLE 6a
Composition of Test Sample:

| | Assay, % (as dried) | | | | | | | | |
|---|---|---|---|---|---|---|---|---|---|
| Sample | Reb | Reb | Reg | Reb | Reb | Reb | Reb | Reb | Reb |
| ID | E | AM | O | D | N | M | H | I | A |
| Reb AM | 0.23 | 99.30 | 0.00 | 0.00 | 0.00 | 0.00 | 0.00 | 0.05 | 0.00 |
| Sample 1 | | | | | | | | | |

TABLE 6b

| | Assay, % (as dried) | | | | | | | |
|---|---|---|---|---|---|---|---|---|
| Sample | | | | | | | | |
| ID | Stev | Reb F | Reb C | Dul. A | Rubu | Reb B | Sbio | TSG |
| Reb AM | 0.00 | 0.00 | 0.00 | 0.00 | 0.00 | 0.00 | 0.26 | 99.84 |
| Sample 1 | | | | | | | | |

TABLE 7
Physical Properties of Reb AM:

| Physical Description | Material & Method | Reb AM Results |
|---|---|---|
| Form | Visual Evaluation | Powder |
| Appearance | Visual Evaluation | Very Fine |
| Odor | Olfactory | Odorless |
| | Evaluation | |
| Color | Visual Evaluation | White |
| Moisture Content | | |

### Solution Stability:

Solubility characteristics were measured as follows. Prepare the following solutions in water and stir at 700 rpm for each. Add heat if necessary at 2 minutes and 30 seconds of stirring. Using a stopwatch, determine how long it takes all powder to dissolve completely and record the temperature at which it dissolves. The following table summarizes the solubility characteristics of Rebaudiosides D, M, and AM. Surprisingly, Reb AM shows significantly higher solubility than other minor and major steviol glycosides.

TABLE 8
Comparison of Solubility Characteristics

| | | Dissolution | Dissolution in | | | Comment |
|---|---|---|---|---|---|---|
| | Test | water | water | Solution | Solution | on |
| Product | Conc. | (Ambient) | (Heated) | after 24 hrs | after 48 hrs | solubility |
| Reb D | 0.05% | Added heat at | 8 min 30 sec | Clear | Clear | Easily Soluble |
| | | 2 minutes/ | temp: 39 deg. C. | | | |
| | | 30 seconds. | | | | |
| Reb D | 0.1% | Added heat at | 15 min 4 sec | Clear | Clear | Easily Soluble |
| | | 2 minutes/ | temp: 72 deg. C. | | | |
| | | 30 seconds. | | | | |
| Reb D | 0.3% | Added heat at | 20 min 27 | Precipitate in | | Requires |
| | | 2 minutes/ | seconds temp: | less than | | dispersion |
| | | 30 seconds. | 78.5 deg. C. | 24 hrs | | agent |
| Reb M | 0.1% | 12 minutes of | No heat needed | Clear | Clear | Easily Soluble |
| | | agitation | | | | |
| Reb M | 0.3% | Added heat at 2 | Heated to 99 deg C. | Clear | Slight | Moderately |
| | | min 30 seconds. | with agitation | | precipitation | Soluble |
| Reb M | 0.5% | Added heat at | Heated to | Moderate | — | Requires |
| | | 2 min 30 sec | 87 deg. C. with | Precipitation | | dispersion |
| | | | agitation. 16 min | in 2 hours | | agent |
| | | | 12 seconds | | | |
| Reb AM | 1% | Stirred for 3 min | No heat needed | Clear | Clear | Easily Soluble |
| | | 28 sec | | | | |
| Reb AM | 5% | Added heat at | 15 min 32 sec at | Clear | Clear | Easily Soluble |
| | | 2 minutes/ | temperature: | | | |
| | | 30 seconds. | 45 C. | | | |
| Reb AM | 10% | Added heat at | 10 min 2 sec | Clear | Slight | Moderately |
| | | 2 minutes/ | Temperature: | | precipitation | soluble |
| | | 30 seconds. | 54 C. | | | |

TABLE 9
Summary of Solution Stability of Major
and Minor steviol glycosides:

| SG/Property | Reb A* | Stevioside* | Reb AM | Reb D | Reb M |
|---|---|---|---|---|---|
| Solubility | <0.7% | <0.7% | 10% | 0.1% | 0.3% |

*Solubility of Stevioside was slightly lower than Reb A in aqueous solution. Ref: Celaya et al (2016). Int. J. of Food Studies, V.5, p 158-166

### Example 10

Reb AM was evaluated for its sensory attributes.

### Sensory Attributes

Steviol glycoside molecules are known for their varied sweetness profiles, which are a function of the sugar moieties present in their structures. Since steviol glycosides contain hydrophobic (steviol) and hydrophilic (sugar moieties), they can display flavor modification at a certain dosage level without contributing any significant detectable sweetness perception.

Isosweet Determination of Reb AM and other Steviol glycosides:
Five concentration levels of Test sweetener were identified to match 2.5%, 5%, 7.5% and 10% sucrose-equivalent in acidified water (pH of 3.2), for which a panel of 40 participants was recruited to conduct two alternate forced choice (2-AFC) test at each concentration level.
Samples were evaluated and isosweet point was determined at a point in which 50% of the panelist selected sucrose sample as sweeter and 50% selected Stevia sample as sweeter
A Beidler model was used to fit the concentration-response relationship using the four isosweet concentrations and their corresponding target sweetness values as the data.
Sweetness potency is calculated as a ratio of sugar concentration to sweetness equivalent. As an example, Reb AM was evaluated.

TABLE 10
Iso-sweet concentration (ppm) and Sweetness Potency (x sugar equivalent)
of Reb AM and other steviol glycosides

| Sweetness Equivalent (ppm) in Water (sweetened | | | | | Sweetness Potency in Water (sweetened | | | |
|---|---|---|---|---|---|---|---|---|
| to achieve designated % SE @ pH = 3.2) | | | | | to achieve x % SE @ pH = 3.2) | | | |
| sugar concentration | 2.5% | 5.0% | 7.5% | 10.0% | 2.5% | 5.0% | 7.5% | 10.0% |
| Reb A | 94 | 299 | NA | NA | 266 | 167 | NA | NA |
| Delta (Reb D) | 62 | 212 | 500 | 926 | 403 | 236 | 150 | 108 |
| PCS-4000 (Reb M) | 84 | 209 | 418 | 832 | 298 | 239 | 179 | 90 |
| Reb AM (Reb AM) | 150 | 365 | 869 | 1750 | 167 | 137 | 86 | 57 |

### Effect of Reb AM on Taste & Flavor Profiles of Food and Beverage Applications

A series of experiments were performed to evaluate the effect of Reb AM on taste and flavor profile. The sweetness and taste/flavor modification can influence each other in food and beverage applications. To determine the influence of the taste and flavor modification in different applications, the FEMA (Flavor and Extract Manufacturing Association) prescribes a sensory method that determines the sweetness perception threshold determination presented in Experiment 1, which is discussed below.

Experiment 1 provides the estimate of Reb AM concentration in water that barely contributes to sweetness perception. The sweetness perception threshold concentration provides significantly less sweetness than 1.5% sugar aqueous solution. The summary of sweetness perception threshold for selected steviol glycosides is below in Table 11.

TABLE 11

| | | | FEMA | FEMA GRAS |
|---|---|---|---|---|
| Steviol | Sweetness Perception | | GRAS | Publication Reference |
| Glycosides | Threshold Concentration | | No | (FEMA Website) |
| Reb A | 30 | ppm | 4601 | GRAS Flavoring |
| | | | | Substances 24 (2008) |
| Reb D | 32.5 | ppm | 4921 | GRAS Flavoring |
| | | | | Substances 29 (2018) |
| Reb M | 24 | ppm | 4922 | GRAS Flavoring |
| | | | | Substances 29 (2018) |
| Reb AM | 50 | ppm | NA | NA |

Experiment 2, which is further discussed below, explores the effect of Reb AM on the flavor profile of a non-alcoholic beverage. A commercial Raspberry Watermelon Coconut Water sample was used without (control) and with Reb AM (test) to determine the effect of Reb AM on different taste attributes of the beverage. The results indicated the test sample having Reb AM had significantly higher mango peach flavor, coconut water flavor, and overall liking compared to the control samples (at 95% confidence).

Experiment 3, which is further discussed below, explores the effect Reb AM on taste & flavor profile of a sweetened dairy product. A sensory panel tested samples of Stevia (Reb A) sweetened, no-sugar-added chocolate flavored dairy protein shake without (control) and with Reb AM. The panel found the test sample containing 50 ppm of Reb AM to be significantly lower bitterness, metallic note, whey protein and lower bitter aftertaste than the control (at 95% confidence) and higher in cocoa flavor, dairy notes, vanilla flavor, and overall liking (at 95% confidence).

A group of trained and experienced taste panel members evaluated no-calorie Lemon-lime carbonated soft drink (CSD) sweetened with 500 ppm of Reb AM, Reb D, or Reb M samples. The panel members found the CSD with Reb AM is less sweet but has significantly less bitterness and sweetness lingering compared to other samples, especially the CSD sweetened with Reb M.

### Experiment 1 of Example 10

### Sweetness Perception Threshold with Reb AM

### Application: Neutral Water

The sweetness perception of 1.5% sugar solution and different solutions of Reb AM were tested with a sensory panel and found that 50 ppm of Reb AM solution in water provided sweetness perception significantly lower than that of 1.5% sugar solution. Therefore we selected 50 ppm of Reb AM as the recognition threshold concentration.

### Methodology

TABLE 12

| | |
|---|---|
| Nature of Participants: | Trained panel |
| Number of Sessions | 1 |
| Number of Participants: | 30 |
| Test Design: | 2- AFC, Balanced, randomized within pair. |
| | Blind |
| Sensory Test Method: | Intensity ratings |
| Environmental Condition | Standard booth lighting |
| Attributes and Scales: | Which sample is sweeter? |
| Statistical Analysis: | Paired comparison Test |
| Sample Size | ~1.5 oz. in a clear capped plastic cup |
| Serving Temperature | Room temperature (~70° F.) |
| Serving/Panelists | Samples served simultaneously. Panelists |
| Instruction: | instructed to read ingredient statement, evaluate |
| | each sample. |

The following table (Table 13) shows an evaluation of the recognition threshold concentration to follow the methodology outlined in section 1.4.2 of the “Guidance for the Sensory Testing of Flavorings with Modifying Properties within the FEMA GRASTM Program”, issued by FEMA (Flavor and Extract Manufacturers Association https://www.femaflavor.org/).

TABLE 13
DATA: n = 30

| Two-Tailed Analysis Table | | | | | |
|---|---|---|---|---|---|
| Report for Result Reb AM | | | | | |
| | | | Percent | Binomial Distribution | |
| | 1.5% | 30 ppm | Frequency | Probability | |
| | Sucrose | Reb AM | Sample 1 | P-value | Sig |
| PC | 29 | 1 | 96.7% | 0.0001 | *** |
| % Frequency | 96.7% | 3.3% | | | |

DATA: n = 30

| Two-Tailed Analysis Table | | | | | |
|---|---|---|---|---|---|
| Report for Result Reb AM | | | | | |
| | | | Percent | Binomial Distribution | |
| | 1.5% | 50 ppm | Frequency | Probability | |
| | Sucrose | Reb AM | Sample 1 | P-value | Sig |
| PC | 23 | 7 | 76.7% | 0.01 | *** |
| % Frequency | 76.7% | 23.3% | | | |

DATA: n = 30

| Two-Tailed Analysis Table | | | | | |
|---|---|---|---|---|---|
| Report for Result ISO3026A | | | | | |
| | | | Percent | Binomial Distribution | |
| | 1.5% | 70 ppm of | Frequency | Probability | |
| | Sucrose | Reb AM | Sample 1 | P-value | Sig |
| PC | 9 | 21 | 30.0% | 0.05 | *** |
| % Frequency | 30% | 70% | | | |

DATA: n = 30

| Two-Tailed Analysis Table | | | | | |
|---|---|---|---|---|---|
| Report for Result Reb AM | | | | | |
| | | | Percent | Binomial Distribution | |
| | 1.5% | 100 ppm | Frequency | Probability | |
| | Sucrose | Reb AM | Sample 1 | P-value | Sig |
| PC | 3 | 27 | 10% | 0.0001 | *** |
| % Frequency | 10% | 90% | | | |

### Experiment 2 of Example 10

### Raspberry Watermelon Coconut Water with Reb AM

### Application: Non-Alcoholic Beverage

### Summary

Thirty panel members evaluated two samples of raspberry watermelon flavored coconut water for overall acceptance and attribute intensities (sweetness, Raspberry flavor, watermelon flavor, coconut water flavor, saltiness, bitterness, and sweet aftertaste, bitter aftertaste) in two sessions. In session one, the two samples included: 1) store-bought Raspberry Watermelon Coconut Water control sample and 2) store-bought Raspberry Watermelon Coconut Water test sample containing Reb AM. The objective of the test was to determine if the addition of Reb AM affects the flavor profile of a non-alcoholic beverage. The results indicated the test sample Reb AM had significantly higher mango peach flavor, coconut water flavor, and overall liking compared to the control samples (at 95% confidence).

### Objective

The project objective is to assess if the addition of Stevia extract solids has an effect on key flavor attributes in various beverage applications.

### Test Objective

The test objective is to determine if the flavor profile and overall acceptance of a Control sample of flavored coconut water differs from a Test sample of the same beverage containing Reb AM.

### Methodology

TABLE 14

| | |
|---|---|
| Nature of Participants: | Trained panel |
| Number of Sessions | 1 |
| Number of Participants: | 30 |
| Test Design: | Balanced, randomized within pair. Blind |
| Sensory Test Method: | Intensity and acceptance ratings |
| Environmental Condition | Standard booth lighting |
| Attributes and Scales: | |

Overall Acceptance on a 10-pt hedonic scale where 10 = Extremely
Like and 0 = Extremely Dislike
Overall liking, sweetness, raspberry flavor, watermelon flavor, coconut
water flavor, astringency, artificial chemical note, bitterness, and
sweet aftertaste, bitter aftertaste. 10-pt continuous intensity scale
where 0 = Imperceptible and 10 = Extremely Pronounced

| | |
|---|---|
| Statistical Analysis: | ANOVA (by Block) with Post Hoc Duncan's |
| | Test |
| Sample Size | ~1.5 oz. in a clear capped plastic cup |
| Serving Temperature | Refrigerated temperature (~45° F.) |
| Serving/Panelists | Samples served simultaneously. Panelists |
| Instruction: | instructed to read ingredient statement, evaluate |
| | each sample. |

### Samples

TABLE 15
Beverage Type I, Non-alcoholic

| | Reference | Reb AM |
|---|---|---|
| *Coconut water raspberry watermelon juice | 100 | 99.995 |
| Reb AM | | 0.005 |
| Total (g) | 100 | 100 |

*Vita Coco store brand

### Results

Table 16 (below) summarizes the overall acceptance and mean attribute intensity results for each sample.

TABLE 16
Mean Scores Raspberry Watermelon Coconut Water with 50 ppm Reb AM
Summary of Mean-Scores, P-Values, and Significance
Test Result Code: Coconut Water (raspberry/watermelon
flavor) Reb AM at 50 ppm
This test was performed on 30 panelists.

| | | Coconut water | | |
|---|---|---|---|---|
| | Coconut water | with 50 ppm of | | |
| Attribute | control | Reb AM | P-Value | Sig |
| Sweet Intensity | 4.38 | 4.44 | 0.6990 | NS |
| Bitter Intensity | 0.32 | 0.24 | 0.4267 | NS |
| Astringency | 1.04 | 1.10 | 0.4942 | NS |
| Coconut Flavor | 4.89 | 5.11 | 0.4372 | NS |
| Watermelon Flavor | b | a | 0.0221 | *** |
| | 3.85 | 4.41 | | |
| Raspberry Flavor | 0.68 | 0.95 | 0.2423 | NS |
| Artificial/Chemical | 2.94 | 2.55 | 0.2583 | NS |
| Note | | | | |
| Sweet Aftertaste | a | b | 0.0905 | ** |
| | 1.60 | 1.33 | | |
| Bitter Aftertaste | 0.36 | 0.29 | 0.5409 | NS |
| Overall Liking | b | a | 0.0710 | ** |
| | 4.49 | 5.04 | | |

* = 80% CI,
** = 90% CI,
*** = 95% CI

The results indicate the test sample Reb AM had significantly higher watermelon flavor and overall liking compared to the control samples (at 95% confidence). Test sample Reb AM had significantly lower sweet aftertaste intensity compared to the control samples (at 90% confidence).

### Conclusion

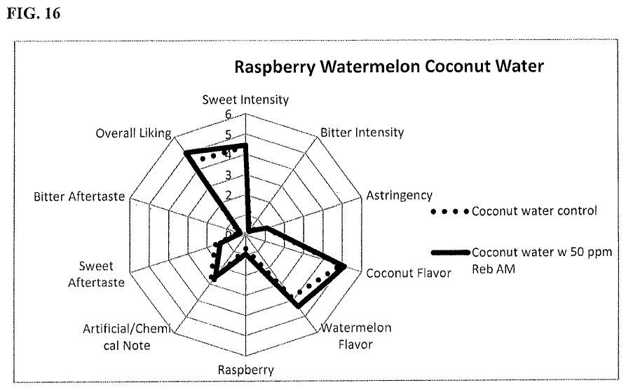

Thirty panelists evaluated two samples of Raspberry Watermelon flavored coconut water for overall acceptance and attribute intensities (sweetness, watermelon flavor, raspberry flavor, coconut water flavor, astringency, artificial/chemical note, bitterness, and sweet aftertaste, bitter aftertaste) in two sessions. In session one, the two samples included: 1) store-bought Raspberry Watermelon Coconut Water control sample and 2) store-bought Raspberry Watermelon Coconut Water test sample containing Reb AM. The objective of the test was to determine if the addition of Reb AM affects the flavor profile of a non-alcoholic beverage. The results indicated the test sample Reb AM had significantly higher watermelon flavor and overall liking compared to the control samples (at 95% confidence). A graph of the results is shown in FIG. 16.

Test sample Reb AM had significantly lower sweet aftertaste intensity compared to the control samples (at 90% confidence).

### Experiment 3 of Example 10

### Chocolate Protein Shake with Reb AM

### Application: Milk/Dairy Product

### Summary

Thirty trained panelists evaluated two samples of chocolate flavored dairy protein shake for overall acceptance and attribute intensities (cocoa flavor, dairy note, whey protein, vanilla, metallic, sweetness, bitterness and aftertaste). The two samples included: 1) no sugar added “Control” sample containing 300 ppm PureCircle Reb A and 2) no sugar added “Test” sample containing 300 ppm PureCircle Reb A and 50 ppm Reb AM. The objective of the test was to determine if the addition of Reb AM affects the flavor profile of a milk product. The panel found the test sample containing 50 ppm of Reb AM to be significantly lower bitterness, metallic note, whey protein and lower bitter aftertaste than the control (at 95% confidence) and higher in cocoa flavor, dairy notes, vanilla flavor, and overall liking (at 95% confidence). Further, there was no significant impact on sweetness intensity.

### Objective

The project objective is to assess if the addition of Stevia extract solids has an effect on key flavor attributes in various beverage applications.

### Test Objective

The test objective is to determine if the flavor profile and overall acceptance of a control sample of dairy beverage application differs from a Test sample of the same beverage containing Reb AM.

### Methodology

TABLE 17

| | |
|---|---|
| Nature of Participants: | Trained panel |
| Number of Sessions | 1 |
| Number of Participants: | 30 |
| Test Design: | Balanced, randomized within pair. Blind |
| Sensory Test Method: | Intensity and acceptance ratings |
| Environmental Condition | Standard booth lighting |
| Attributes and Scales: | |

Overall Acceptance on a 10-pt hedonic scale where 10 = Extremely
Like and 0 = Extremely Dislike
Overall Liking, sweetness, bitterness, cocoa flavor, dairy notes,
chocolate, whey protein notes, metallic note, vanilla note, and
Aftertaste. 10-pt continuous intensity scale where 0 =
Imperceptible and 10 = Extremely Pronounced

| | |
|---|---|
| Statistical Analysis: | ANOVA (by Block) with Post Hoc Duncan's |
| | Test |
| Sample Size | ~1.5 oz. in a clear capped plastic cup |
| Serving Temperature | Refrigerated temperature (~45° F.) |
| Serving/Panelists | Samples served simultaneously. Panelists |
| Instruction: | instructed to read ingredient statement, |
| | evaluate each sample. |

### Samples

TABLE 18

| | Sugar | 50 ppm |
|---|---|---|
| Ingredient list | Reference | Reb AM |
| Milk, 2% | 86.47 | 86.465 |
| Whey Protein 90 Instant - Non GMO | 6.8250 | 6.8250 |
| (Prod: 18618) | | |
| Non-Fat Dry Milk | 4.6269 | 4.6269 |
| Maltrin QD M585 | 1.1066 | 1.1066 |
| Vitamin Blend - | 0.0063 | 0.0063 |
| Xanthan Gum (Cold dissolve) | 0.0359 | 0.0359 |
| Forbes 10/12 Cocoa powder 7113 | 0.7194 | 0.7194 |
| Vanilla Flavor Powder | 0.1799 | 0.1799 |
| Reb A | 0.0300 | 0.0300 |
| Reb AM | | 0.0050 |
| TOTAL | 100 | 100 |

| | Sugar | 165 ppm |
|---|---|---|
| Sugar Contribution (grams) per 100 grams* | Reference | Reb AM |
| Milk, 2% | 4.08 | 4.15 |
| Non-Fat Dry Milk | 2.41 | 2.41 |
| Maltrin QD M585 | 0.08 | 0.08 |
| TOTAL | 8.07 | 6.64 |

*Calculated with Genesis R&D version 11.4

TABLE 19
Effect Reb AM on flavor modification of Chocolate Protein shake
Summary of Mean-Scores, P-Values, and Significance
Test Result Code: PROTEIN6
Test Description: Chocolate Vanilla Protein Dairy Shake: 50 ppm Reb AM
This test was performed on 30 panelists.

| | Control - NSA | Test - NSA Protein | | |
|---|---|---|---|---|
| | Protein Shake | Shake w Reb A & | | |
| Attribute | w/Reb A | 50 ppm Reb AM | P-Value | Sig |
| Sweet Intensity | 6.04 | 5.98 | 0.7329 | NS |
| Bitterness | a | b | 0.0138 | *** |
| | 1.98 | 1.46 | | |
| Metallic Note | a | b | 0.0311 | *** |
| | 1.93 | 1.48 | | |
| Cocoa Flavor | b | a | 0.0409 | *** |
| | 4.06 | 4.55 | | |
| Dairy Note | b | a | 0.0515 | ** |
| | 4.10 | 4.59 | | |
| Whey Protein | a | b | 0.0460 | *** |
| Note | 4.79 | 4.32 | | |
| Vanilla Note | b | a | 0.0174 | *** |
| | 2.10 | 2.52 | | |
| Sweet Aftertaste | 1.82 | 1.65 | 0.2130 | NS |
| Bitter Aftertaste | a | b | 0.0495 | *** |
| | 1.03 | 0.77 | | |
| Overall Liking | b | a | 0.0001 | *** |
| | 4.80 | 5.59 | | |

* = 80% CI,
** = 90% CI,
*** = 95% CI

The panel found the test sample containing 50 ppm of Reb AM to be significantly lower bitterness, metallic note, whey protein and lower bitter aftertaste than the control (at 95% confidence).

The panel found the test sample containing 50 ppm of Reb AM to be significantly higher in cocoa flavor, dairy notes, vanilla flavor, and overall liking (at 95% confidence).

### Conclusion

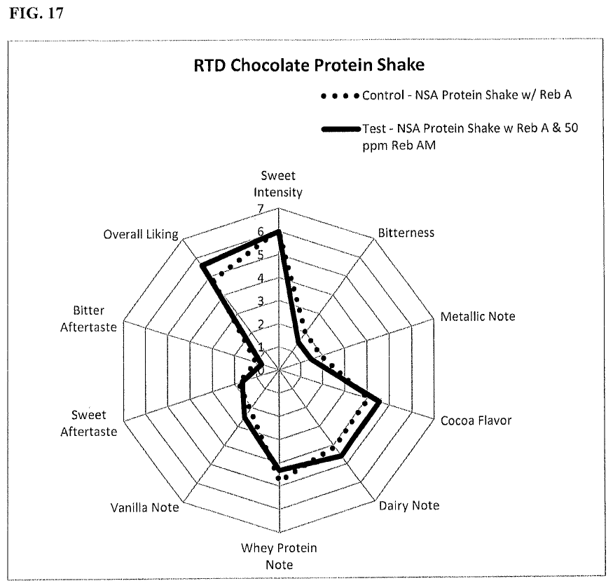

Thirty panelists evaluated two samples of chocolate flavored dairy protein shake for overall acceptance and attribute intensities (cocoa flavor, dairy note, whey protein, vanilla, metallic, sweetness, bitterness and aftertaste). The two samples included: 1) no sugar added “Control” sample containing 300 ppm PureCircle Reb A and 2) no sugar added “Test” sample containing 300 ppm PureCircle Reb A and 50 ppm Reb AM. The objective of the test was to determine if the addition of Reb AM affects the flavor profile of a milk product. The panel found the test sample containing 50 ppm of Reb AM to be significantly lower bitterness, metallic note, whey protein and lower bitter aftertaste than the control (at 95% confidence) and higher in cocoa flavor, dairy notes, vanilla flavor, and overall liking (at 95% confidence). Further, there was no significant impact on sweetness intensity. A graph of the results is shown in FIG. 17.

Although the invention and its advantages have been described in detail, it should be understood that various changes, substitutions and alterations can be made herein without departing from the spirit and scope of the invention as defined by the appended claims. Moreover, the scope of the application is not intended to be limited to the particular embodiments of the invention described in the specification. As one of ordinary skill in the art will readily appreciate from the disclosure of the invention, the compositions, processes, methods, and steps, presently existing or later to be developed that perform substantially the same function or achieve substantially the same result as the corresponding embodiments described herein may be utilized according to the invention.

We claim:

1. A method for enhancing flavor in a consumable product, comprising adding highly purified Rebaudioside AM to the product at a level below a sweetness detection level of Rebaudioside AM, wherein Rebaudioside AM has the formula:

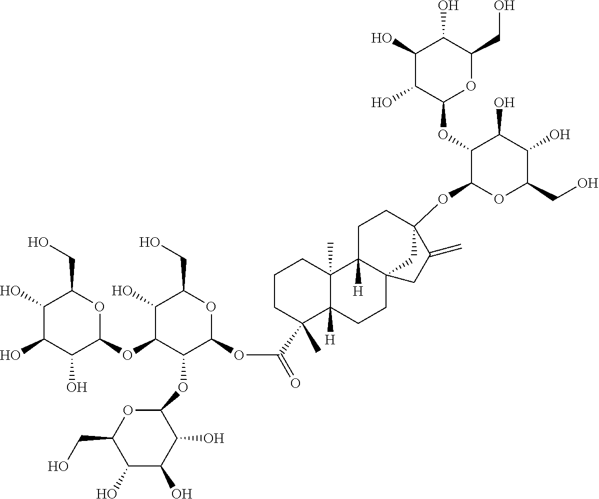

2. A method for producing the highly purified rebaudioside AM of claim 1, comprising the steps of:
a. providing a starting composition comprising an organic compound with at least one carbon atom;
b. providing a biocatalyst selected from the group consisting of an enzyme preparation, a cell or a microorganism; said biocatalyst comprising at least one enzyme capable of converting the starting composition to rebaudioside AM;
c. contacting the biocatalyst with a medium containing the starting composition to produce a medium comprising rebaudioside AM

3. The method of claim 2 further comprising the step of:
d. separating the rebaudioside AM from the medium to provide a highly purified rebaudioside AM composition.

4. The method of claim 2, wherein the starting composition is selected from the group consisting of steviol, steviolmonoside, steviolmonoside A, steviolbioside, steviolbioside A, steviolbioside B, rubusoside, stevioside, stevioside A (rebaudioside KA), stevioside B, stevioside C, rebaudioside E, rebaudioside E2, rebaudioside E3, other steviol glycosides, polyols, carbohydrates, and combinations thereof.

5. The method of claim 2, wherein the microorganism is selected from the group consisting of E. coli, Saccharomyces sp., Aspergillus sp., Pichia sp., Bacillus sp., and Yarrowia sp.

6. The method of claim 2, wherein the enzyme is selected from the group consisting of: a steviol biosynthesis enzyme, a UDP glucosyltransferase, a UDP glucose recycling enzyme, a mevalonate (MVA) pathway enzyme, a 2-C-methyl-D-erythritol-4-phosphate pathway (MEP/DOXP) enzyme, geranylgeranyl diphosphate synthase, copalyl diphosphate synthase, kaurene synthase, kaurene oxidase, kaurenoic acid 13-hydroxylase (KAH), steviol synthetase, deoxyxylulose 5-phosphate synthase (DXS), D-1-deoxyxylulose 5-phosphate reductoisomerase (DXR), 4-diphosphocytidyl-2-C-methyl-D-erythritol synthase (CMS), 4-diphosphocytidyl-2-C-methyl-D-erythritol kinase (CMK), 4-diphosphocytidyl-2-C-methyl-D-erythritol 2,4-cyclodiphosphate synthase (MCS), 1-hydroxy-2-methyl-2(E)-butenyl 4-diphosphate synthase (HDS), 1-hydroxy-2-methyl-2(E)-butenyl 4-diphosphate reductase (HDR), acetoacetyl-CoA thiolase, truncated HMG-CoA reductase, mevalonate kinase, phosphomevalonate kinase, mevalonate pyrophosphate decarboxylase, cytochrome P450 reductase, UGT74G1, UGT85C2, UGT91D2, EUGT11, UGTS12, UGT76G1, or mutant variant thereof having >85% amino-acid sequence identity, >86% amino-acid sequence identity, >87% amino-acid sequence identity, >88% amino-acid sequence identity, >89% amino-acid sequence identity, >90% amino-acid sequence identity, >91% amino-acid sequence identity, >92% amino-acid sequence identity, >93% amino-acid sequence identity, >94% amino-acid sequence identity, >95% amino-acid sequence identity, >96% amino-acid sequence identity, >97% amino-acid sequence identity, >98% amino-acid sequence identity, >99% amino-acid sequence identity; and combinations thereof.

7. The method of claim 3, wherein the rebaudioside AM content in highly purified rebaudioside AM composition is greater than about 95% by weight on a dry basis.

8. A consumable product made by the method of claim 1, wherein the product is selected from the group consisting of a food, a beverage, a pharmaceutical composition, a tobacco product, a nutraceutical composition, an oral hygiene composition, and a cosmetic composition.

9. The consumable product of claim 8, further comprising at least one additive selected from the group consisting of carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, flavonoids, alcohols, polymers and combinations thereof.

10. The consumable product of claim 8, further comprising at least one functional ingredient selected from the group consisting of saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

11. The consumable product of claim 8, further comprising a compound selected from the group consisting of rebaudioside A, rebaudioside A2, rebaudioside A3, rebaudioside B, rebaudioside C, rebaudioside C2, rebaudioside D, rebaudioside D2, rebaudioside E, rebaudioside E2, rebaudioside E3, rebaudioside F, rebaudioside F2, rebaudioside F3, rebaudioside G, rebaudioside H, rebaudioside I, rebaudioside 12, rebaudioside 13, rebaudioside J, rebaudioside K, rebaudioside K2, rebaudioside KA, rebaudioside L, rebaudioside M, rebaudioside M2, rebaudioside N, rebaudioside O, rebaudioside O2, rebaudioside Q, rebaudioside Q2, rebaudioside Q3, rebaudioside R, rebaudioside S, rebaudioside T, rebaudioside T1, rebaudioside U, rebaudioside U2, rebaudioside V, rebaudioside W, rebaudioside W2, rebaudioside W3, rebaudioside Y, rebaudioside Z1, rebaudioside Z2, dulcoside A, dulcoside C, rubusoside, steviolbioside, steviolbioside A, steviolbioside B, steviolmonoside, steviolmonoside A, stevioside, stevioside A, stevioside B, stevioside C, stevioside D, stevioside E, stevioside E2, stevioside F, NSF-02, Mogroside V, Luo Han Guo, allulose, D-allose, D-tagatose, erythritol, brazzein, neohesperidin dihydrochalcone, glycyrrhizic acid and its salts, thaumatin, perillartine, pernandulcin, mukuroziosides, baiyunoside, phlomisoside-I, dimethyl-hexahydrofluorene-dicarboxylic acid, abrusosides, periandrin, carnosiflosides, cyclocarioside, pterocaryosides, polypodoside A, brazilin, hernandulcin, phillodulcin, glycyphyllin, phlorizin, trilobatin, dihydroflavonol, dihydroquercetin-3-acetate, neoastilibin, trans-cinnamaldehyde, monatin and its salts, selligueain A, hematoxylin, monellin, osladin, pterocaryoside A, pterocaryoside B, mabinlin, pentadin, miraculin, curculin, neoculin, chlorogenic acid, cynarin, siamenoside, sucralose, potassium acesulfame, aspartame, alitame, saccharin, cyclamate, neotame, dulcin, suosan advantame, gymnemic acid, hodulcin, ziziphin, lactisole, glutamate, aspartic acid, glycine, alanine, threonine, proline, serine, lysine, tryptophan, maltitol, mannitol, sorbitol, lactitol, xylitol, inositol, isomalt, propylene glycol, glycerol, threitol, galactitol, hydrogenated isomaltulose, reduced isomalto-oligosaccharides, reduced xylo-oligosaccharides, reduced gentio-oligosaccharides, reduced maltose syrup, reduced glucose syrup, hydrogenated starch hydrolyzates, polyglycitols, sugar alcohols, L-sugars, L-sorbose, L-arabinose, trehalose, galactose, rhamnose, various cyclodextrins, cyclic oligosaccharides, various types of maltodextrins, dextran, sucrose, glucose, ribulose, fructose, threose, xylose, lyxose, altrose, mannose, idose, lactose, maltose, invert sugar, isotrehalose, neotrehalose, isomaltulose, erythrose, deoxyribose, gulose, talose, erythrulose, xylulose, cellobiose, amylopectin, glucosamine, mannosamine, glucuronic acid, gluconic acid, glucono-lactone, abequose, galactosamine, beet oligosaccharides, isomalto-oligosaccharides (isomaltose, isomaltotriose, panose and the like), xylo-oligosaccharides (xylotriose, xylobiose and the like), xylo-terminated oligosaccharides, gentio-oligosaccharides (gentiobiose, gentiotriose, gentiotetraose and the like), nigero-oligosaccharides, palatinose oligosaccharides, fructooligosaccharides (kestose, nystose and the like), maltotetraol, maltotriol, malto-oligosaccharides (maltotriose, maltotetraose, maltopentaose, maltohexaose, maltoheptaose and the like), starch, inulin, inulo-oligosaccharides, lactulose, melibiose, raffinose, isomerized liquid sugars such as high fructose corn syrups, coupling sugars, soybean oligosaccharides, D-psicose, D-ribose, L-glucose, L-fucose, D-turanose, D-leucrose.
